(19) United States
(12) Patent Application Publication
(10) Pub. No.: US 2026/0193640 A1
(43) Pub. Date: Jul. 9, 2026

(54) SCREENING AND CHARACTERIZATION OF BINDING REAGENTS UNDER EQUILIBRIUM CONDITIONS

(71) Applicant: Nautilus Subsidiary, Inc., Seattle, WA (US)

(72) Inventor: Kara JUNEAU, San Carlos, CA (US)

(21) Appl. No.: 19/414,808

(22) Filed: Dec. 10, 2025

Related U.S. Application Data
(60) Provisional application No. 63/730,911, filed on Dec. 11, 2024.

(51) Int. Cl. C12N 15/10

(52) U.S. Cl. CPC C12N 15/1065

(57) ABSTRACT

Methods and systems are provided for selecting binding reagents from a library and characterizing selected binding reagents for assays. A selected binding reagent may bind to two or more differing analytes, or epitopes thereof. Further provided are systems that facilitate selection and/or characterization of binding reagents under assay conditions.

### CROSS-REFERENCE

This application claims priority to U.S. Provisional Application No. 63/730,911, filed on Dec. 11, 2024, which is incorporated herein by reference in its entirety.

### BACKGROUND

Selection methods for the generation of affinity reagents are typically designed to select for binding reagents with high affinity and specificity for a single epitope or protein. For some applications, it may be useful to select binding reagents which bind multiple epitopes, or to characterize the binding patterns of binding reagents which are not specific for a single protein or epitope. These promiscuous affinity reagents can provide advantages for combinatorial methods of identifying a large variety of different analytes, such as proteins, using a relatively small variety of affinity reagents. See, for example, U.S. Pat. Nos. 10,473,654 and 11,970,693, U.S. Patent Publication No. 2023/0114905, and Egertson, et al. BioRxiv (2021), DOI: 10.1101/2021.10.11.463967, each of which is incorporated herein by reference. Furthermore, some research or clinical applications benefit from affinity reagents that demonstrate high avidity, increased avidity being correlated with reduced dissociation rate.

### SUMMARY

In an aspect, provided herein is a method of selecting a binding reagent having a binding specificity for an epitope Θ, comprising: (a) providing a plurality of binding targets, wherein each binding target of the plurality of binding targets comprises the epitope Θ, and wherein each binding target of the plurality of binding targets is co-localized with a unique binding target barcode moiety, (b) providing a library of binding reagents wherein each binding reagent of the library of binding reagents differs from each other binding reagent of the library of binding reagents with respect to binding specificity, and wherein each binding reagent of the library of binding reagents is attached to a unique binding reagent barcode moiety, (c) combining the plurality of binding targets with the library of binding reagents, thereby coupling binding reagents of the library of binding reagents to binding targets of the plurality of binding targets, (d) forming a plurality of interaction moieties, wherein each interaction moiety of the plurality of interaction moieties comprises information from a binding target barcode moiety of a binding target of the plurality of binding targets and information from the binding reagent barcode moiety, and (e) detecting two or more interaction moieties containing information from a same binding reagent barcode moiety, thereby selecting the binding reagent of the library of binding reagents having a binding specificity for the epitope Θ.

In another aspect, provided herein is a method of characterizing a binding reagent having a binding specificity for an epitope Θ, comprising: (a) providing a plurality of binding targets, wherein each binding target of the plurality of binding targets is derived from a proteome, wherein a first fraction of binding targets of the plurality of binding targets comprises the epitope Θ, wherein a second fraction of binding targets of the plurality of binding targets does not comprise the epitope Θ, and wherein each unique binding target of the plurality of binding targets is co-localized with a unique binding target barcode moiety, (b) contacting to the plurality of binding targets a plurality of binding reagents, wherein each binding reagent of the plurality of binding reagents is substantially identical, wherein each binding reagent of the plurality of binding reagents has a binding specificity for the epitope Θ, and wherein each individual binding reagent of the plurality of binding reagents is attached to a binding reagent barcode moiety, (c) forming a plurality of interaction moieties, wherein each interaction moiety of the plurality of interaction moieties comprises information from a binding target barcode moiety of the plurality of binding targets and information from the binding reagent barcode moiety, (d) detecting each interaction moiety of the plurality of interaction moieties, and (e) determining for each unique binding target barcode moiety a quantity of interaction moieties of the plurality of interaction moieties containing the information from the unique binding target barcode moiety.

In another aspect, provided herein is a system for characterizing a binding reagent, comprising: a) a vessel comprising a plurality of binding reagents, b) a plurality of binding targets, c) a polymer sequencing device, and d) a fluidic system that is configured to transfer the plurality of binding reagents from the vessel to the plurality of binding targets, and is further configured to transfer binding interaction moieties from the plurality of binding targets to the polymer sequencing device.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A, 1B, 1C, and 1D illustrate steps of a method for screening a library of binding reagents utilizing a plurality of binding targets, in accordance with some embodiments. FIGS. 1E, 1F, 1G, and 1H illustrate steps of a method of characterizing a binding reagent utilizing a plurality of binding targets, in accordance with some embodiments.

FIGS. 2A, 2B, 2C, 2D, and 2E depict methods of forming interaction moieties, in accordance with some embodiments.

FIGS. 3A, 3B, 3C, and 3D display configurations of a method that occurs in a fluid phase that utilizes a suspensible solid support, in accordance with some embodiments.

FIGS. 4A and 4B show methods of recording information in an interaction moiety that utilize enzymatic systems, in accordance with some embodiments.

FIG. 5 illustrates a schematic of a system configured to perform certain methods set forth herein, in accordance with some embodiments.

### DETAILED DESCRIPTION

Binding reagents, including common affinity reagents such as antibodies, antibody fragments, aptamers, and mini-protein binders, have numerous uses, including in vitro uses (e.g., biochemical or diagnostic assays), and in vivo uses (e.g., therapeutic uses). A useful binding reagent is often identified through a screening process that selects the binding reagent with a desired binding affinity or specificity from amongst a pool of varied binding reagents. Binding reagents may be further subjected to a maturation process, in which a selected binding reagent is further varied to optimize its binding affinity or specificity for one or more binding targets. A binding reagent can also undergo a characterization process that determines both on-target and off-target binding affinity and/or specificities, as well as a tendency of the binding reagent to participate in orthogonal binding interactions with assay components other than candidate binding targets (often referred to as “non-specific binding”).

Some in vitro applications may utilize more complex binding reagents. Complexity may be added, for example, by attaching detectable labels (e.g., fluorophores, luminophores, barcode tags, purification tags, etc.) to an affinity reagent. Some binding reagents may further comprise a linking moiety (e.g., a polymer strand) or a retaining moiety (e.g., a branched polymer, a dendrimer, a nucleic acid nanoparticle, an inorganic nanoparticle) that attaches detectable labels or other moieties (e.g., a tether strand) to an affinity reagent. In particular cases, a binding reagent may be formed by complexing together multiple affinity reagents, optionally with other components (e.g., detectable labels, linking moieties, retaining moieties, tether strands, etc.).

By forming a more complex binding reagent from an affinity reagent or a binding ligand, the characterized binding affinity and/or specificity of a binding reagent containing the affinity reagent may become altered. Screening and selection processes are often performed utilizing affinity reagents rather than more complex binding reagents. When selected affinity reagents are subsequently incorporated into more complex binding reagents, their affinity and/or specificity may be observed to differ from their simpler forms. Without wishing to be bound by theory, the variation in binding behavior between a singular affinity reagent or binding ligand and a more complex binding reagent containing the affinity reagent or binding ligand may be due to the increased chemical complexity of the binding reagent. Addition of components to a binding reagent (e.g., a linking moiety, a retaining moiety, a detectable label, a barcode tag, a purification tag, a tether strand, etc.) can decrease the binding affinity and/or specificity of an affinity reagent or binding ligand for an on-target binding interaction or increase the binding affinity and/or specificity of the affinity reagent for an off-target or orthogonal binding interaction. Likewise, formation of a multivalent binding reagent (i.e., a binding reagent containing multiple affinity reagents-see for example U.S. Pat. No. 11,692,217B2, which is herein incorporated by reference in its entirety) can increase the binding affinity and/or specificity of an affinity reagent due to an avidity effect.

The characterization of binding reagents may be particularly important in single-molecule systems where interactions between a single binding reagent and a single binding target can be observed. In bulk- or ensemble-scale observations, off-target or orthogonal binding interactions may be observed as a background signal against a more dominant signal from on-target binding. For single-molecule observations, individual interactions between binding reagents and on-target binding targets and off-target binding targets can be individually observed, facilitating a more complex understanding of the binding dynamics of a binding reagent. Single-molecule characterization of a binding reagent against a varied group of binding targets may provide information on both the likelihood of the binding reagent binding to any particular binding target of the group of binding targets, as well as the binding affinity of that binding interaction.

Accordingly, it may be preferable to screen and/or characterize a binding reagent in an in vitro format that more closely resembles the in vitro format in which the binding reagent will be utilized. For example, if binding reagents are to be utilized in an assay that observes binding of a binding reagent at equilibrium, it may be preferable to characterize the binding specificity and/or affinity of the binding reagent in an equilibrium format. Likewise, if binding reagents are to be utilized in an assay that occurs on an array of binding targets, it may be preferable to characterize the binding specificity and/or affinity of the binding reagent against an array of binding targets. Alternatively, if binding reagents are to be utilized in an assay that occurs in a fluid phase, it may be preferable to characterize the binding specificity and/or affinity of the binding reagent in the fluid phase.

Provided herein are methods and system for screening and/or characterizing the binding specificity and/or affinity of one or more affinity reagents when at least one affinity reagent is provided as a multi-component binding reagent. The methods and systems may be provided in an array-based format, in which binding reagents and binding targets form bound complexes on the surface of a solid support. Alternatively, the methods and system may be provided in a fluid-phase format, in which binding reagents and binding targets form bound complexes in the fluid phase. The methods and system may utilize interaction moieties (e.g., nucleic acid barcodes, peptide barcodes) that facilitate detection of binding reagents that formed binding interactions with binding targets. Methods may include detection of interaction moieties and interpretation of information contained within the interaction moieties to determine which binding targets and binding reagents formed binding interactions. Information contained in interaction moieties can include whole or partial barcode sequences (e.g., binding target barcode sequences, binding reagent barcode sequences) or other detectable chemical structures that, when detected, allow identification of a binding target and/or binding reagent involved in a binding interaction that gave rise to the interaction moiety.

It will be recognized that methods described herein of recording binding interactions via barcode-based methods may be equally applicable to methods of screening libraries of binding reagents for binding reagents having a particular binding specificity and methods of characterizing the binding specificity of binding reagents across broad panels of possible binding targets.

### Definitions

Terms used herein will be understood to take on their ordinary meaning in the relevant art unless specified otherwise. Several terms used herein and their meanings are set forth below.

In some of the implementations described herein, the term “address,” when used in reference to an array, can mean a location in an array occupied by, or configured to be occupied by, a particular molecule or analyte such as a protein, nucleic acid, structured nucleic acid particle or reactive moiety. An address can contain a single analyte molecule, or it can contain a population of several analyte molecules of the same species (i.e. an ensemble of the molecules). Alternatively, an address can include a population of molecules that are different species such as an attached analyte and a linked binding reagent. Addresses of an array are typically discrete. The discrete sites can be contiguous, or they can have interstitial spaces between each other. An array useful herein can have, for example, addresses that are separated by less than 100 microns, 10 microns, 1 micron, 0.5 micron, 0.1 micron, 0.01 micron or less. Alternatively or additionally, an array can have addresses that are separated by at least 0.01 micron, 0.1 micron, 0.5 micron, 1 micron, 10 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁷, 1×10⁸, 1×10⁹, 1×10¹⁰, 1×10¹¹, 1×10¹², or more addresses, some or all of which are occupied by analytes or molecules. An address that is configured to bind an analyte, for example by the presence of attachment moieties, is referred to herein as a “site.”

In some of the implementations described herein, the term “affinity reagent” can refer to a molecule or other substance that is capable of specifically or reproducibly binding to an analyte (e.g. protein) or moiety (e.g. post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include chemically reactive affinity reagents, catalytic affinity reagents (e.g., kinases, proteases, etc.) or chemically non-reactive affinity reagents (e.g., antibodies or fragments thereof). An affinity reagent can be chemically non-reactive and non-catalytic, thereby not permanently altering the chemical structure of an analyte to which it binds. Affinity reagents that can be particularly useful for binding to proteins include, but are not limited to, antibodies such as full-length antibodies or functional fragments thereof (e.g., Fab′ fragments, F(ab′)₂ fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

In some of the implementations described herein, the term “antibody” can refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some of the implementations described herein, the term “anchoring moiety” can refer to a molecule or particle that serves as an intermediary for attaching a binding target to a surface (e.g., a solid support or a microbead). An anchoring group may be covalently or non-covalently attached to a surface and/or a polypeptide. An anchoring group may be a biomolecule, polymer, particle, nanoparticle, or any other entity that is capable of attaching to a surface or polypeptide. In some cases, an anchoring group may be a structured nucleic acid particle.

In some of the implementations described herein, the term “array” can refer to a population of analytes (e.g. proteins) that are co-localized with unique identifiers such that the analytes can be distinguished from each other. A unique identifier can be a solid support (e.g. particle or bead), a particle (e.g., a structured nucleic acid particle (SNAP)), address on a solid support, tag, label (e.g. luminophore), or barcode (e.g. nucleic acid barcode) that is co-localized with an analyte and that is distinct from other identifiers in the array. Analytes can be co-localized with unique identifiers by attachment, for example, via covalent or non-covalent bonds. An array can include different analytes that are each attached to different unique identifiers. An array can include different unique identifiers that are attached to the same or similar types of analytes. An array can include separate solid supports, or separate sites on a given solid support, that each bear a respective analyte, wherein the respective analytes can be identified according to the locations of the solid supports or sites. Analytes that can be included in an array can be, for example, nucleic acids such as structured nucleic acid particles, proteins, enzymes, glycans, affinity reagents, ligands, or receptors.

In some of the implementations described herein, the terms “barcode” and “barcode moiety” can refer synonymously to a polymer strand comprising a plurality of residues, in which the identity of each residue can independently vary from the identity of each other residue of the polymer strand, and in which the sequence of residues of the polymer strand is detectable. The quantity of possible sequences encodable in a barcode can depend upon the quantity of residues in the barcode and the quantity of distinguishable options for each residues. For example, a barcode comprising deoxyribonucleic acid can be composed of the four naturally occurring nucleotides, so a permutation of every possible sequence for a DNA barcode of length n nucleotides may be 4″. Likewise, a barcode composed of the 20 naturally-occurring amino acids having a length of n amino acids can be arranged in 20″ unique sequences. A barcode may comprise a plurality of polymerized monomers, including biological monomers such as ribonucleotides, deoxyribonucleotides, amino acids, saccharides, modified or non-natural analogs thereof, and synthetic polymer monomers, such as olefins, acrylates, and glycols.

In some of the implementations described herein, the term “binding affinity” or “affinity” can refer synonymously to the strength or extent of binding between a binding reagent and a binding target. The binding affinity of a binding reagent for a binding target may be qualified as being “high affinity,” “medium affinity,” or “low affinity.” A binding affinity of a binding reagent for a binding target, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

In some of the implementations described herein, the term “binding interaction” can refer to a reaction that associates a binding reagent to an analyte. A binding interaction may be a covalent or non-covalent interaction. A binding interaction may associate a binding reagent to an analyte for a sufficient length of time to detect a complex formed by the binding reagent and analyte.

In some of the implementations described herein, the term “binding reagent” can refer to an entity that is capable of reproducibly binding to a binding target or other substance. A binding reagent can comprise an affinity agent or a plurality thereof. A binding reagent can comprise a binding ligand such as a small molecule compound (e.g., a molecule of <1000 Dalton molecular weight), a metabolite, a polysaccharide, or a peptide. A binding reagent may be detectable if one or more detectable labels (e.g., fluorophores, luminophores, barcode moieties) are attached or otherwise incorporated with the binding reagent. A binding reagent can further comprise a linking group or linking moiety that couples components (e.g., affinity agents, detectable labels) of a binding reagent together. A linking group or linking moiety may comprise a nanoparticle, such as a nucleic acid nanoparticle, or a non-nucleic acid nanoparticle (e.g., a polymer nanoparticle, a semiconductor nanoparticle, a carbon nanoparticle, a metal nanoparticle). For a binding reagent comprising an antibody affinity reagent, a linking moiety can include an antibody-binding protein such as Protein A, Protein G, or a secondary antibody. A binding reagent may comprise one or more additional moieties that mediate the interaction of the binding reagent with a binding target or other entity, such as one or more pendant polymer strands.

In some of the implementations described herein, the term “binding specificity” can refer to the tendency of an affinity reagent to preferentially interact with a binding partner or epitope relative to other binding partners or epitopes. An affinity reagent may have a calculated, observed, known, or predicted binding specificity for any possible binding partner or epitope. Binding specificity may refer to selectivity for a single binding partner, epitope, or target moiety in a sample over all, some, or at least one other analyte in the sample. Moreover, binding specificity may refer to selectivity for a subset of binding partners, epitopes, or target moieties in a sample over at least one other analyte in the sample.

In some of the implementations described herein, the term “binding target” can refer to a molecule or moiety that is a candidate for coupling with a binding reagent. A binding target can comprise an epitope that can be bound by a binding reagent. A binding target can be a naturally-occurring molecule or moiety, such as a biopolymer (e.g., a protein, a glycoprotein, a nucleic acid, a polysaccharide, a lipid, etc.). A binding target can be an artificial or synthesized molecule or moiety, such as an inorganic nanoparticle, a carbon nanoparticle, a synthetic polymer, a mineral particle, a ceramic particle, a metal particle, a semiconductor particle, etc. A binding target can comprise a biopolymer attached to an artificial or synthetic molecule or moiety, such as a peptide attached to a synthetic polymer.

In some of the implementations described herein, the term “co-localized” can refer to two or more entities with a minimally varying spatial and/or temporal proximity. Two entities may be co-localized if they are both immobilized at a same address of a solid support. Two entities may be co-localized if their respective addresses on a solid support cannot be optically resolved from each other. Two entities may be co-localized if they are both attached to a same linking moiety. Two entities may be co-localized if their respective mass transfer through a fluid phase is not independent of each other. For example, two unbound entities in a fluid phase may be co-localized if they are joined by a linking moiety such that the entities diffusion paths become coupled over a sufficiently long time period.

In some of the implementations described herein, the term “common,” when used in reference to a molecule, can refer to a moiety that is present in a substantially identical form in two or more molecules or entities. For example, two proteins with differing primary amino acid sequences can contain a common epitope if each of the two proteins contains a same shorter amino acid sequence within their differing primary amino acid sequences.

The term “comprising” is intended herein to be open-ended, including not only the recited elements, but further encompassing any additional elements.

In some of the implementations described herein, the term “each,” when used in reference to a collection of items, is intended to identify an individual item in the collection but does not necessarily refer to every item in the collection. Exceptions can occur if explicit disclosure or context clearly dictates otherwise.

In some of the implementations described herein, the term “epitope” can refer to a molecule or part of a molecule, which is recognized by or binds specifically to a binding reagent or paratope. Epitopes may include amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, miniprotein or other affinity reagent. An epitope can optionally bind an antibody to elicit an immune response. However, an epitope need not necessarily participate in, nor be capable of, eliciting an immune response.

In some of the implementations described herein, the term “flanking sequence,” when used in reference to a binding target, can refer to one or more residues adjacent to an epitope of the binding target. For a polymeric binding target in a linearized, extended, or denatured conformation, a flanking sequence may comprise one or more residues contiguous with an epitope of the binding target. For a polymeric binding target in a folded, coiled, or otherwise compacted conformation, a flanking sequence can include one or more residues contiguous with an epitope of the binding target, and/or one or more non-contiguous residues brought into proximity of the epitope by the conformation of the binding target. A flanking sequence can comprise one or more residues in close enough proximity to an epitope of a binding target to contact an epitope-binding region of an affinity reagent (e.g., a complementarity-determining region of an antibody).

In some of the implementations described herein, the term “fluid-phase” or “fluid phase,” when used in reference to a molecule or particle participating in a binding interaction, can mean the molecule or particle is in a state wherein it is mobile in a fluid, for example, being capable of diffusing through the fluid to another molecule or particle that is complementary to the molecule or particle in the binding interaction. A fluid-phase molecule or particle may be attached to a solid support provided the moiety that attaches the molecule or particle to the solid support permits sufficient movement of the molecule or particle through the fluid to contact another molecule or particle that is complementary to the molecule or particle in a binding interaction. A fluid-phase molecule or particle may be attached to a solid support by a linker, such as a flexible linker. An immobilized analyte that is provided in a denatured or partially-denatured state may contain one or more fluid-phase portions of its molecular structure.

In some of the implementations described herein, the term “immobilized,” when used in reference to a molecule or particle that is in contact with a fluid phase, can refer to the molecule or particle or a portion thereof being prevented from diffusing in the fluid phase. For example, immobilization can occur due to confinement at, or attachment to, a solid phase. Immobilization can be temporary (e.g. for the duration of one or more steps of a method set forth herein) or permanent. Immobilization can be reversible or irreversible under conditions utilized for a method, system or composition set forth herein. A denatured molecule may be considered immobilized if the molecule as a whole cannot diffuse through the fluid phase, even if portions of the molecular structure have an ability to diffuse in regions of the fluid adjacent to the immobilization site of the molecule.

In some of the implementations described herein, the term “interaction moiety” can refer to a detectable molecule or moiety formed by the coupling of a binding reagent to a specific binding target, in which the detectable molecule or moiety contains information on the specific binding target to which the binding reagent was coupled. An interaction moiety can comprise a binding target barcode moiety or a portion thereof. An interaction moiety can comprise information, such as a code or sequence, that is obtained from a binding target barcode moiety. An interaction moiety can further comprise a binding reagent barcode moiety or a portion thereof, or information that is obtained from the binding reagent barcode moiety. An interaction moiety can remain detectable after the dissociation of a complex containing a binding reagent and a binding target. Information included in an interaction moiety can include a detectable moiety that is appended to a molecule when a binding interaction occurs. For example, a binding target barcode moiety can be biotinylated by a biotin ligase enzyme when a binding target co-localized with the binding target barcode moiety is bound by a binding reagent containing the biotin ligase enzyme.

In some of the implementations described herein, the terms “label” and “detectable label” can refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., sequencing of, or hybridization to, a nucleic acid barcode) or in a time-resolved manner (e.g., time-resolved fluorescence). A label may produce a signal with a characteristic frequency, intensity, polarity, duration, wavelength, sequence, or fingerprint.

In some of the implementations described herein, the term “library,” when used in reference to a plurality of binding reagents, can refer to the plurality of binding reagents comprising two or more binding reagents with differing binding specificities. A library of binding reagents can comprise two or more binding reagents with differing structures. A library of binding reagents can comprise two or more differing affinity reagents. The binding reagents of a library of binding reagents may be uncharacterized with respect to the binding specificity of each unique binding reagent of the library of binding reagents. A library of binding reagents can comprise two or more unique binding reagents as characterized by binding specificity, with two or more copies of each unique binding reagent being present in the library of binding reagents. A library of binding reagents can comprise two or more binding reagents that are degenerate with respect to binding specificity, i.e., two structurally differing binding reagents with substantially similar binding specificity.

In some of the implementations described herein, the terms “linker” and “linking moiety” can refer synonymously to a moiety that connects two objects to each other. One or both objects can be a molecule (e.g. affinity reagent or analyte), solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, polypeptide, nucleotide, nucleic acid, nucleic acid origami, dendrimer, peptide nucleic acid (PNA), polysaccharide, carbon, nitrogen, oxygen, ether, sulfur, or disulfide. A linker can be a bead or particle such as a structured nucleic acid particle.

In some of the implementations described herein, the term “moiety” can refer to a component or part of a molecule. The term does not necessarily denote the relative size of the component or part compared to the rest of the molecule, unless indicated otherwise. A moiety can include one or more atoms.

In some of the implementations described herein, the term “paratope” can refer to a molecule or part of an affinity reagent, which recognizes or binds to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

In some of the implementations described herein, the term “particle” can mean an object having a largest dimension between 10 nm and 1 mm. The object can be composed of a rigid or semi-rigid material. The particle can be insoluble in a fluid such as aqueous liquid. A particle can have a shape characterized, for example, as a sphere, ovoid, polyhedron, or other recognized shape whether having regular or irregular dimensions. Exemplary particles include, but are not limited to, structured nucleic acid particles (SNAPs) such as nucleic acid origami particles; optically detectable particles such as fluorescent nanoparticles, FluoSpheres™, and quantum dots; organic particles; inorganic particles; viral particles, such as phage particles having analytes displayed on their surfaces; gel particles; or particles made from solid support materials set forth herein or known in the art.

In some of the implementations described herein, the term “polymer” can refer to a molecule having a plurality of monomer subunits connected via a network of covalent bonds. The network may contain a single type of monomer subunit, or two or more types of monomer subunits. A polymer network may have a pattern of subunits or a random network of subunits (e.g., a protein). The network can include one or more chain(s) of the monomer subunits. A polymer can be linear or branched. A linear polymer includes only one chain in the network of covalent bonds. A branched polymer includes at least two chains in the network of covalent bonds. For example, a branched polymer can include at least 2, 3, 4, 5, 6, 8, 10 or more chains in the network of covalent bonds. Alternatively or additionally, a branched polymer can include at most 10, 8, 6, 5, 4, 3 or 2 chains in the network of covalent bonds. A polymer can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

In some of the implementations described herein, the terms “protein” and “polypeptide” can refer synonymously to a molecule including three or more amino acids joined by peptide bonds. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

In some of the implementations described herein, the term “recognize” can refer to the capability of two or more molecules to interact with each other through non-covalent bonding such as hydrogen bonding, metal coordination, hydrophobic forces, van der Waals forces, π-π interactions, halogen bonding, or resonant interaction effects.

In some of the implementations described herein, the term “retaining component” can refer to a particle, molecule or material to which one or more moieties of an affinity reagent or analyte are attached. Exemplary retaining components include, but are not limited to, structured nucleic acid particles, nucleic acid origami, particles made of solid support materials, or polymers such as branched polymers or dendrimers. Affinity reagent moieties that can be attached to a retaining component, directly or indirectly, include for example, one or more paratopes, one or more labels, one or more antibodies, one or more nucleic acid aptamers, one or more nucleic acid tags or the like.

In some of the implementations described herein, the term “sequence context,” when used in reference to a binding target, can refer to the one or more flanking sequences of an epitope of the binding target. A plurality of binding targets may comprise a plurality of sequence contexts if there is variability of flanking sequences with respect to a common epitope of the binding targets. For example, a plurality of peptide binding targets may comprise a plurality of sequence contexts if the peptide binding targets each comprise a common amino acid epitope but vary with respect to the amino acid sequences of flanking sequences amongst the plurality of peptide binding targets. A plurality of binding targets may comprise a single sequence context if there is no variability of flanking sequences with respect to a common epitope of the binding targets.

In some of the implementations described herein, the term “single,” when used in reference to an object such as an analyte, can mean that the object is individually manipulated or distinguished from other objects. A single object can also be referred to as one, and only one, object. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some of the implementations described herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure (i.e. the helical twist or direction of the polynucleotide strand) of a SNAP can be configured to be more dense than a nucleic acid molecule of similar length in a random coil or other non-structured state. A SNAP can optionally be modified to permit attachment of additional molecules to the SNAP. A SNAP may contain DNA, RNA, PNA, or modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some of the implementations described herein, the term “unique” can refer to a molecule, object, or moiety having a detectable characteristic that distinguishes it from one or more other molecules, objects, or moieties. For example, a first protein may be considered unique from a second protein if the primary amino acid sequence of the first protein differs from the primary amino acid sequence of the second protein. In contrast, a first protein may not be considered unique from a second protein if both proteins have the same primary amino acid sequence. However, two proteins can be considered unique if they both have the same primary amino acid sequence but one or more different modifications (e.g., post-translational modifications, terminal amino acid modifications, etc.). In another example, a first binding reagent may be considered unique from a second binding reagent if the first binding reagent has a different structure than the structure of the second binding reagent. Alternatively, a first binding reagent may be considered unique from a second binding reagent if the first binding reagent has a detectably different binding specificity than the second binding reagent. A plurality of molecules, objects, or moieties can be characterized as containing two or more molecules, objects, or moieties that are unique, and further characterized as containing two or more molecules, objects, or moieties that are substantially identical or indistinguishable.

In some of the implementations described herein, the term “unique identifier” can refer to a moiety, object or substance that is co-localized with an analyte and that is distinct from other identifiers, throughout one or more steps of a process. The moiety, object or substance can be, for example, a solid support such as a particle or bead; a location on a solid support; a site in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to a co-localized analyte, for example, being synthetically attached to the co-localized analyte. Alternatively, a unique identifier can be endogenous to the analyte, for example, being attached or co-localized with the analyte in the native milieu of the analyte.

In some of the implementations described herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

### Methods of Recording Binding Interactions

The present disclosure provides methods for screening libraries of binding reagents to identify a binding reagent having a desired binding specificity. The present disclosure further provides methods of characterizing the binding specificity of individual binding reagents. Certain methods, compositions, and systems for recording the binding interactions of binding reagents during assays may be useful for both screening and characterization assays.

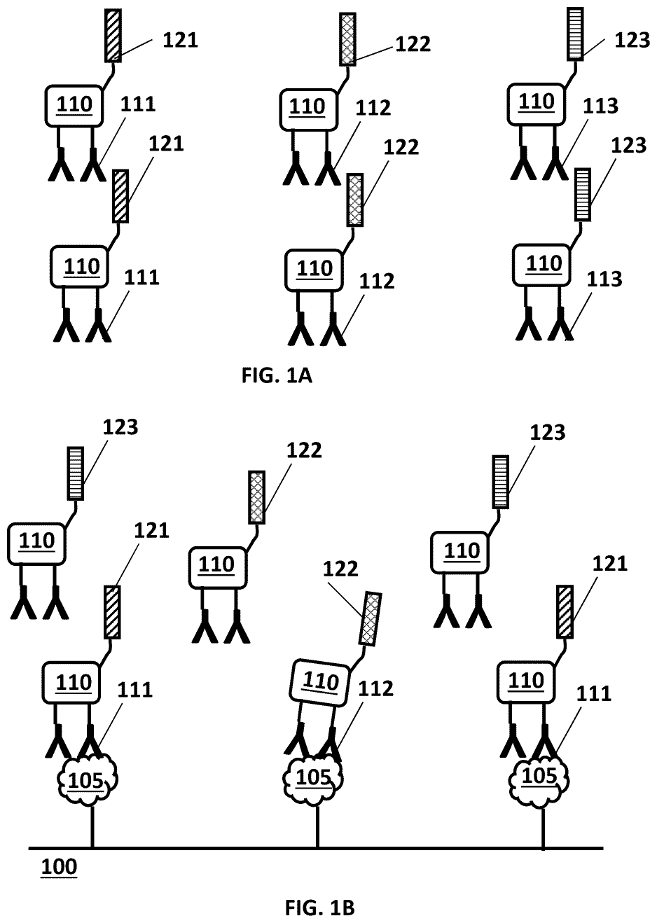

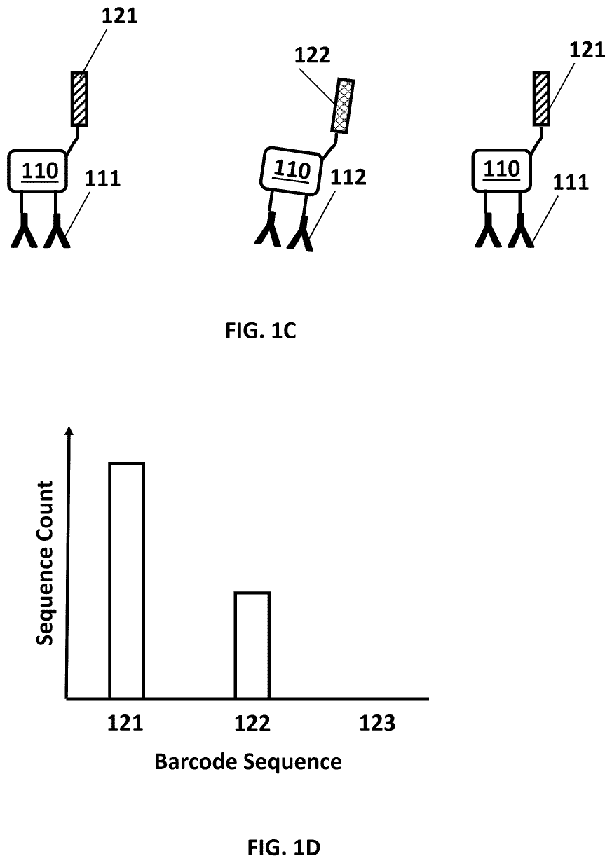

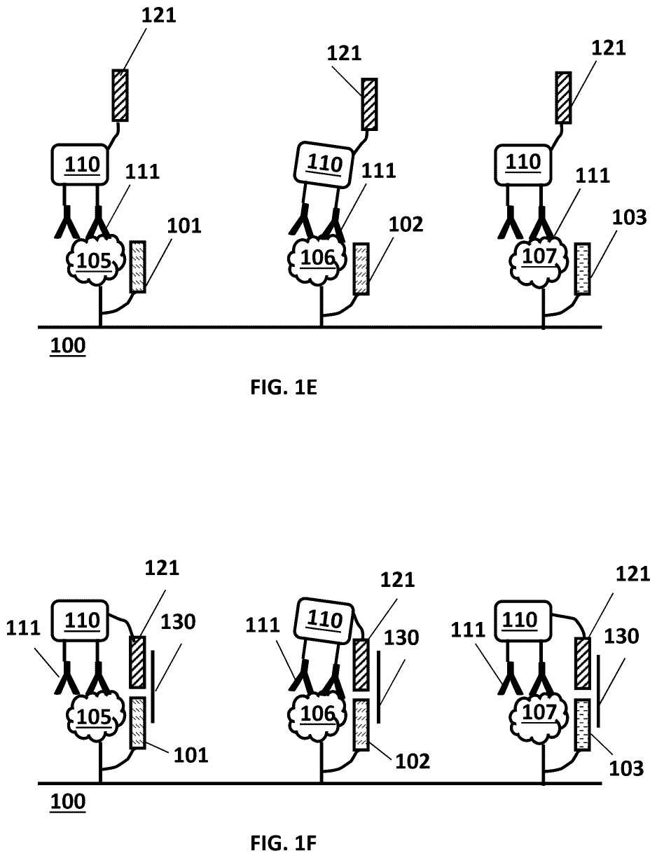

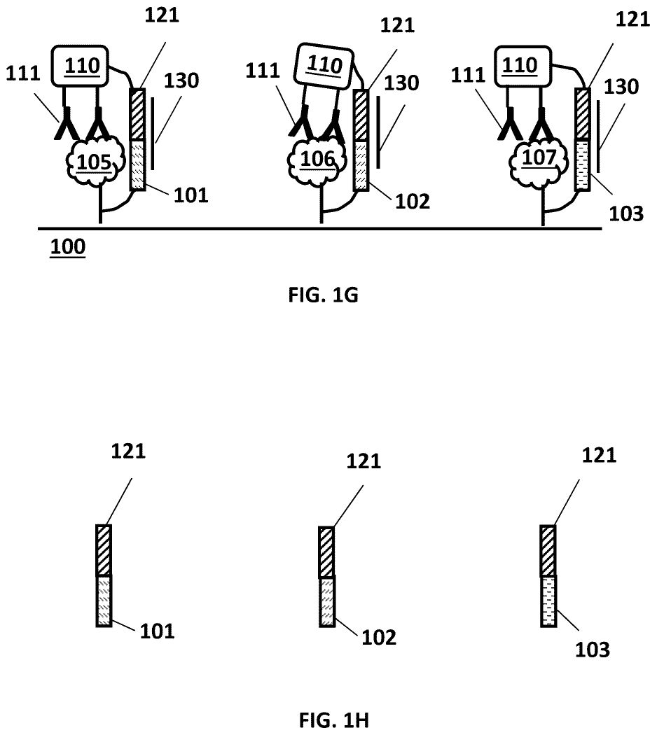

FIGS. 1A-1D illustrate aspects of detecting binding interaction between binding targets and binding reagents. The method described is exemplified with an array of binding targets, but the method may be readily modified to occur with binding targets mobile in a fluid phase. FIG. 1A depicts a plurality of binding reagents. In the configuration shown, each binding reagent comprises one of a plurality of different affinity reagents (111, 112, 113) attached to a retaining moiety 110 (e.g., a nanoparticle, a nucleic acid particle, a polymer particle, a dendrimer, etc.). Each binding reagent further comprises a barcode moiety (121, 122, 123) that is common to any other binding reagent containing affinity reagents of the same specificity (i.e., each binding reagent comprising affinity reagents 111 further contains barcode moiety 121). As shown in FIG. 1A, a plurality of each unique binding reagent (as determined by binding specificity) of the library of binding reagents is present in the library of binding reagents. As shown in FIG. 1B, the plurality of binding reagents has been contacted to a solid support 100 comprising an array of binding targets 105 (e.g., peptides, polypeptides, non-polypeptide analytes, etc.), thereby facilitating binding of binding reagents of the plurality of binding reagents to binding targets 105 of the array of binding reagents 105. In the depicted configuration, binding reagents comprising affinity reagents 111 and 112 bind to the binding targets 105. Unbound binding reagents may be separated from contact with the solid support 100. Optionally, as shown in FIG. 1C, after unbound binding reagents are separated from contact with the solid support 100, the bound binding reagents may be eluted from the solid support 100. After elution, the barcode moieties 121 and 122, or copies thereof, may be collected from the eluted binding reagents. Alternatively, the barcode moieties 121 and 122, or copies thereof, may be collected while the binding reagents remain bound to the array of binding targets 105. The collected barcode moieties 105 may be provided to a sequencing device that detects the sequence of each collected barcode moiety. As shown in FIG. 1D, if a plurality of each binding reagent is provided in the plurality of binding reagents, differing counts of barcode sequences specific to each unique binding reagent may be detected for each unique barcode moiety. In the formed configuration of FIG. 1B and as shown in FIG. 1D, binding reagents containing affinity reagents 111 are observed to bind to binding targets more frequently than binding reagents containing affinity reagents 112, and no binding reagents containing affinity reagents 123 are observed to bind to binding targets. Accordingly, a binding reagent corresponding to a most frequently detected binding reagent barcode sequence may be selected as a binding reagent for an epitope Q, as set forth herein.

FIGS. 1E-1H illustrate aspects of an alternative method of recording binding interactions between binding targets and binding reagents. The method described is exemplified with an array of binding targets, but the method may be readily modified to occur with binding targets mobile in a fluid phase. FIG. 1E depicts a solid support 100 comprising three differing binding targets (105, 106, 107). Each of the three binding targets is co-localized with a unique binding target barcode moiety (101, 102, 103, respectively) that contains a unique code or sequence that is specific to only the array address or binding target with which it is co-localized. Each of the three binding targets is bound by a copy of a binding reagent. Each of the binding reagents comprises one or more affinity reagents 111 and a binding reagent barcode moiety 121, optionally joined by a retaining moiety 110 or a linking moiety. FIG. 1F illustrates a second optional array configuration, in which each pair of binding reagent barcode moieties and binding target barcode moieties is joined by a bridging molecule 130 that couples to the binding reagent barcode moiety 121 and the binding target barcode moiety (101, 102, 103), thereby bringing the individual barcode moieties in a close proximity. In some embodiments set forth herein, interaction moieties are formed without bridging molecules 130. FIG. 1G depicts a third array configuration in which each binding reagent barcode moiety 121 has been ligated (e.g., via nucleic acid ligation, via peptide ligation) to a single binding target barcode moiety (101, 102, or 103). FIG. 1H depicts a plurality of complete barcode moieties comprising a binding reagent barcode moiety 121 and a binding target barcode moiety (101, 102, or 103) after the complete barcode moieties are released from the binding reagent-binding target complexes. Each complete barcode moiety of the plurality of barcode moieties can be detected (e.g., by sequencing, by labeled capture on a barcode array), thereby identifying the presence of a binding interaction between the binding reagent and a specific binding target of the array of binding targets.

The skilled person will readily recognize that the method of FIGS. 1A-1D and the method of FIGS. 1E-1H can readily be readily modified with aspects of the other method. For example, both methods can be configured to include heterogeneous pluralities of binding targets and/or binding reagents. Likewise, both methods can be configured to include homogeneous pluralities of binding targets and/or binding reagents.

A method of characterizing a binding reagent may comprise a step of forming an interaction moiety. An interaction moiety may comprise information from a binding target barcode moiety that is co-localized with a binding target, and optionally may further comprise information from a binding reagent barcode moiety that is co-localized with a binding reagent. In some configurations, an interaction moiety may comprise a code or sequence from a binding target barcode moiety that is co-localized with a binding target, and optionally may further comprise a code or sequence from a binding reagent barcode moiety that is co-localized with a binding reagent. In some configurations, an interaction moiety may comprise only information from a binding reagent barcode moiety.

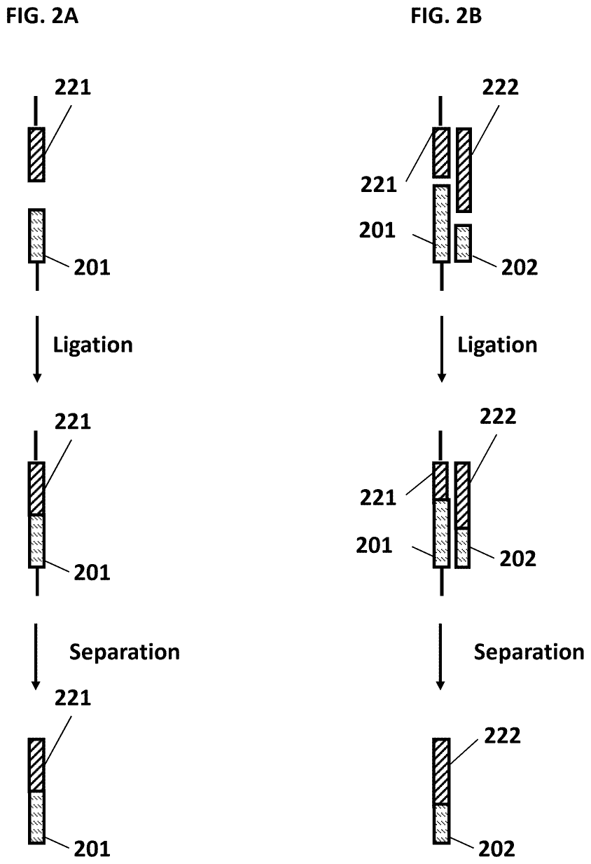

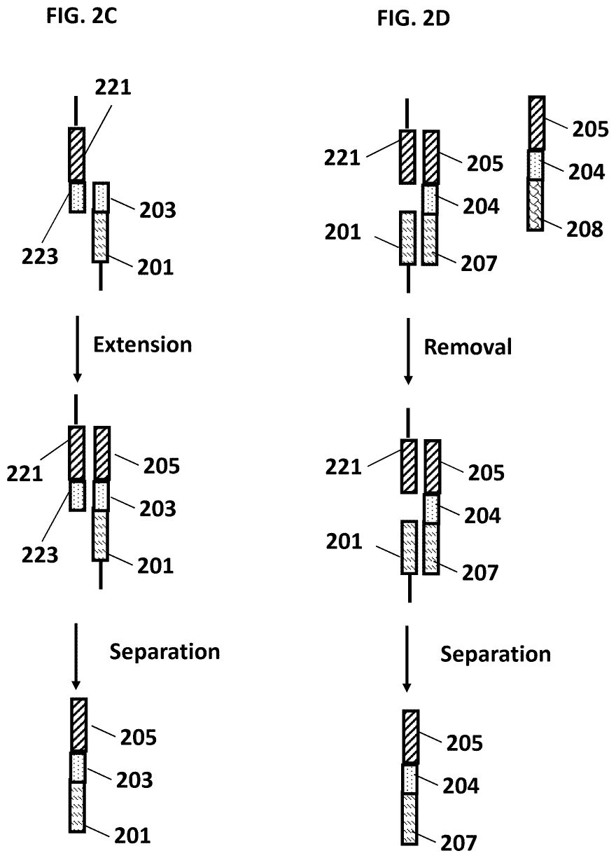

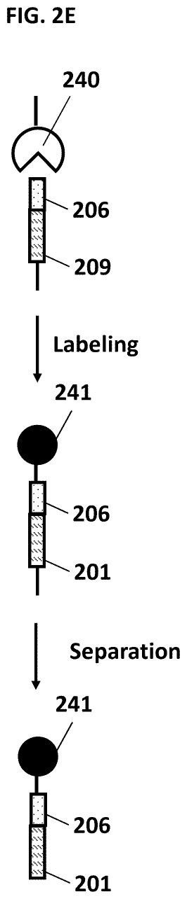

Nucleic acids may be particularly useful for forming an interaction moiety. FIGS. 2A-2D illustrate methods of forming an interaction moiety utilizing nucleic acids. FIG. 2A depicts a method of single-stranded ligation of two nucleic acids to form an interaction moiety. A first single-stranded nucleic acid barcode moiety 201 is brought in proximity with a second single-stranded nucleic acid barcode moiety 221. The first single-stranded nucleic acid barcode moiety 201 is ligated to the second single-stranded nucleic acid barcode moiety 221 chemically or enzymatically, thereby forming an interaction moiety comprising the first single-stranded nucleic acid barcode moiety 201 and the second single-stranded nucleic acid barcode moiety 221. After ligation, the interaction moiety may be separated from a binding reagent-binding target complex to which it is attached. FIG. 2B depicts a method of double-stranded ligation of two nucleic acids to form an interaction moiety. The method shown utilizes overlapping single-stranded nucleic acids, but the method can be readily adapted to blunt end ligation. A first partially single-stranded nucleic acid barcode moiety comprising a short strand 201 and a long strand 222 is brought coupled to a second partially single-stranded nucleic acid barcode moiety comprising a long strand 221 and a short strand 202 by hybridization between the respective long strands 201 and 222. After hybridization, the complex is ligated chemically or enzymatically, thereby forming an interaction moiety comprising the long strand 222 and the short strand 202. After ligation, the interaction moiety can be separated from the binding reagent-binding target complex, for example by dissociating the nucleic acid strand comprising the long strand 222 and the short strand 202.

FIG. 2C depicts a method of forming an interaction moiety by extension of a nucleic acid strand. A first nucleic acid barcode moiety comprises a code or sequence strand 221 and a hybridization sequence 223. A second nucleic acid barcode moiety comprises a code or sequence strand 201 and a hybridization sequence 203. The first nucleic acid barcode moiety is coupled to the second nucleic acid barcode moiety by hybridization of the hybridization sequence 223 to the hybridization sequence 203. Subsequently, either the first nucleic acid barcode moiety or the second nucleic acid barcode moiety may be extended by a polymerase extension reaction (e.g., polymerase chain reaction), thereby forming an interaction moiety (FIG. 2C depicts extension of the second nucleic acid barcode moiety to form the reverse complement 205 of the code or sequence strand 221). Subsequently, the interaction moiety can be separated from the binding reagent-binding target complex, for example by dehybridizing and/or cleaving the interaction moiety.

FIG. 2D depicts the capture of a bridging interaction moiety to detect a binding interaction. A first single-stranded nucleic acid barcode moiety 201 and a second single-stranded nucleic acid barcode moiety 221 are bound by a bridging oligonucleotide comprising a complementary first single-stranded nucleic acid barcode moiety 205 and a second single-stranded nucleic acid barcode moiety 207 joined by a linking moiety 204. The bridging oligonucleotide may be configured to only bind stably when the first single-stranded nucleic acid barcode moiety 201 and the second single-stranded nucleic acid barcode moiety 221 are in a close proximity (i.e., binding of the bridging oligonucleotide to only one of the barcode moieties will not stably bind the bridging oligonucleotide). The coupled nucleic acids are also contacted with an unbound bridging oligonucleotide comprising a complementary first single-stranded nucleic acid barcode moiety 205 and a third single-stranded nucleic acid barcode moiety 208 joined by a linking moiety 204. The unbound bridging oligonucleotide has not bound because the third single-stranded nucleic acid barcode moiety 208 is not complementary to the first single-stranded nucleic acid barcode moiety 201. The unbound bridging oligonucleotide may be removed from contact with the coupled nucleic acids (e.g., by rinsing of the unbound bridging oligonucleotide, by decanting or removal of a fluidic medium containing the unbound bridging oligonucleotide). After removing the unbound bridging oligonucleotide, the bound bridging oligonucleotide may be separated from the binding reagent-binding target complex, thereby providing an interaction moiety comprising the bridging oligonucleotide. Alternatively, the bridging interaction moiety of FIG. 2D can be utilized to form an interaction moiety by a gap fill and ligation method. For example, the second configuration of FIG. 2D can be contacted with a polymerase to extend the first single-stranded barcode moiety 201 or the second single-stranded nucleic acid barcode moiety 221 with the reverse complement of linking moiety 204. After the extension reaction, the complex can be contacted with a ligase enzyme that attaches the first single-stranded barcode moiety 201 to the second single-stranded nucleic acid barcode moiety 221. The ligated moiety can subsequently be separated from the bridging moiety and detached from linking moieties, thereby providing an interaction moiety comprising the first single-stranded barcode moiety 201 and the second single-stranded nucleic acid barcode moiety 221.

In a useful configuration, a bridging moiety or a binding reagent may include a cycle-specific barcode moiety. A cycle-specific barcode moiety can comprise a residue sequence that encodes for a specific detection cycle in which an interaction occurred. For multi-cycle methods, incorporating a barcode moiety into an interaction moiety can facilitate pooling and simultaneous detection of interaction moieties from multiple cycles. Further, a bridging moiety, binding target, or a binding reagent may include a vessel-specific barcode moiety. If pluralities of binding targets or pluralities of binding reagents are distributed into multiple vessels, a vessel specific moiety may facilitate identification of within which vessel an interaction occurred. Additional useful methods and compositions for interaction moieties are provided in U.S. patent application Ser. No. 19/338,819, which is herein incorporated by reference in its entirety.

If only one binding reagent is being characterized, it may not be necessary to incorporate a binding reagent barcode moiety into an interaction moiety. Likewise, if only one binding target is being utilized to screen or characterize a binding reagent, it may not be necessary to incorporate a binding target barcode moiety into an interaction moiety. A method or system utilizing a bridging molecule (like FIG. 2D) can utilize a universal barcode moiety or binding sequence that is present on any characterized binding reagent and binds to the bridging molecule, but does not provide any unique or specific code or sequence. Alternatively, a method of characterizing a binding reagent may be readily multiplexed with respect to the binding reagent (e.g., characterizing two or more binding reagents simultaneously) if each binding reagent is provided a unique barcode moiety that distinguishes it from any other binding reagent. In a multiplexed method, each interaction moiety can comprise a first code or sequence from a binding reagent barcode moiety and a second code or sequence from a binding target barcode moiety, thereby providing information on which binding reagent bound to which binding target.

In some configurations, peptides may be useful for forming an interaction moiety. In some configurations, a binding reagent barcode can comprise a first peptide and a binding target barcode can comprise a second peptide. A method of forming an interaction moiety may comprise ligating the first peptide to the second peptide. FIG. 2E depicts an alternative approach to forming an interaction moiety via barcode labeling of a peptide. A peptide barcode moiety is co-localized with a binding target. The peptide barcode moiety comprises a code or sequence 209 and a labeling peptide 206 (e.g., an Avi-tag). A binding reagent is co-localized with an enzyme 240 (e.g., a biotin ligase enzyme) that is configured to form a label on the labeling peptide 206. The labeling peptide 206 is labeled by the enzyme 240, thereby an interaction moiety comprising the code or sequence 209, the labeling peptide 206, and the label 241. After labeling, the interaction moiety may be separated from the binding target. In a useful configuration, labeling of an Avi-tag by a biotin ligase enzyme may attach a biotin moiety to a peptide barcode. The biotin may facilitate capture of an interaction moiety after separation from a binding target by binding of the biotin label to an avidin or streptavidin moiety (e.g., a streptavidin moiety immobilized on a solid support). Binding target barcode moieties that were not labeled due to absence of binding of a binding reagent comprising a labeling enzyme can be rinsed from the solid support comprising the avidin or streptavidin moiety, thereby retaining only the interaction moiety.

A barcode moiety, as set forth herein, may comprise any suitable polymer strand comprising a sequence of detectable residues. Polypeptides and oligonucleotides are especially useful due to the availability of sequencing systems for these polymer types. The length of a barcode moiety may be determined in part by the type of polymer chosen for a barcode moiety. For example, DNA chains can comprise the four naturally-occurring nucleotides, so the number of unique sequences for a DNA strand of length n nucleotides is 4ⁿ. Over 10⁶ unique nucleotide sequences can be provided for a chain of 10 nucleotides length. Likewise, peptide chains can comprise the twenty naturally-occurring amino acids, so the number of unique sequences for a peptide strand of length n amino acids is 20ⁿ. Over 10⁶ unique amino acid sequences can be provided for a chain of 5 amino acids length. A barcode moiety may have a length of at least about 2, 3, 4, 5, 6, 7, 8, 9, 10, 11, 12, 13, 14, 15, 20, 25, 30, 50, or more than 50 residues (e.g., nucleotides, amino acids). A barcode moiety may have a length of no more than about 50, 30, 25, 20, 15, 14, 13, 12, 11, 10, 9, 8, 7, 6, 5, 4, 3, or less than 3 residues.

After separating an interaction moiety from a binding target or a binding target-binding reagent complex, the interaction moiety may be detected. Detecting the code or sequence from the binding reagent barcode and/or the code or sequence from the binding target barcode for the interaction moiety can comprise sequencing the interaction moiety with a nucleic acid sequencing device or a peptide sequencing device. A sequencing device may be provided an interaction moiety comprising a code or sequence from a binding target barcode moiety. Optionally, the interaction moiety may further comprise a code or sequence from a binding reagent barcode moiety. Optionally, the interaction moiety may further comprise one or more non-coding regions or sequences.

After one or more interaction moieties have been detected, the sequence information may be analyzed and/or categorized to determine which binding targets were bound by a binding reagent. The manner of analysis and categorization may depend upon the configuration of the interaction moieties formed during a method of characterizing a binding reagent. For example, if binding target barcode moieties are cleaved from binding targets regardless of whether a binding target barcode moiety was incorporated into an interaction moiety, then each sequence would be categorized by presence or absence of a code or sequence from a binding reagent barcode moiety. In another example, if two or more binding reagents are characterized in multiplex, then each interaction moiety sequence must be analyzed to determine which binding reagent barcode sequence is present in the interaction moiety.

If interaction moieties are formed to include a binding reagent barcode sequence, determining a set of binding target barcodes associated with the binding reagent barcode can comprise: i) categorizing each interaction moiety sequence according to the presence or absence of the binding reagent barcode, ii) for each interaction moiety sequence having a presence of the binding reagent barcode, identifying the binding target barcode; iii) identifying for each identified binding target barcode a binding target corresponding to the binding target barcode, and iv) adding the binding target to a set of identified binding targets for the binding reagent barcode.

If interaction moieties are formed without a binding reagent barcode sequence (e.g., the method of FIG. 2E), determining a set of binding target barcodes associated with the binding reagent barcode can comprise: i) for each interaction moiety sequence, identifying the binding target barcode; ii) identifying for each identified binding target barcode a binding target corresponding to the binding target barcode, and iii) adding the binding target to a set of identified binding targets for the binding reagent barcode.

In some cases, a binding reagent may interact with a specific binding target two or more times. For example, a plurality of binding targets may comprise two or more identical binding targets, in which each of the two or more identical binding targets form a binding interaction with identical binding reagents of a plurality of binding reagents. A method may further comprise quantifying for each unique binding target a quantity of binding reagents bound to the unique binding target. In some cases, each of two or more binding interactions between a binding target and a binding reagent may form a unique interaction moiety. In other cases, a single binding target barcode moiety or binding reagent barcode moiety may be serially extended to record each binding interaction.

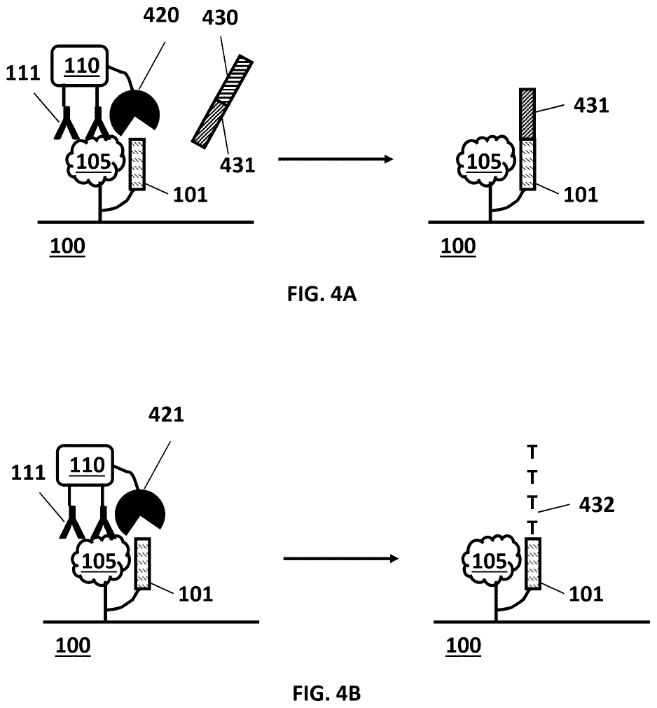

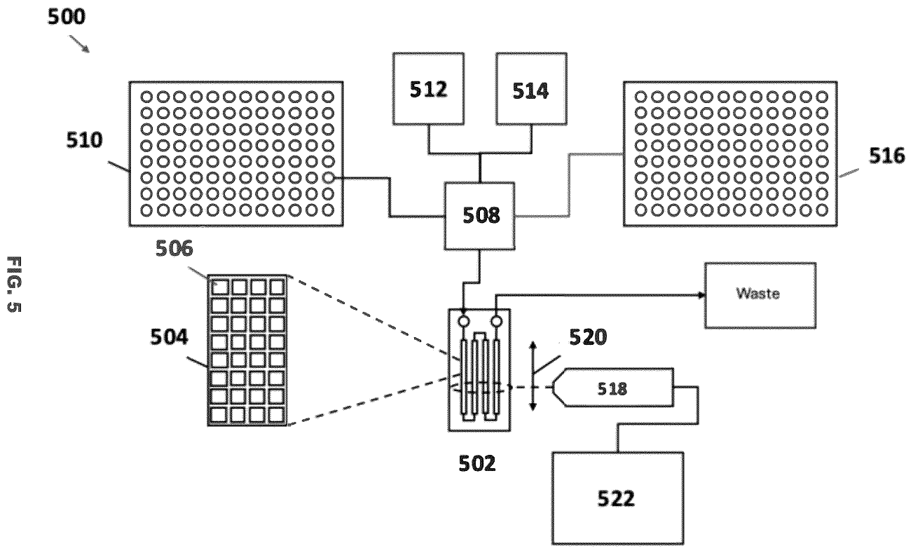

Although certain methods, compositions, and systems have been described for binding reagent screening and/or characterization, in which binding reagents are associated with a binding reagent barcode moiety that contains a specific residue sequence, in other cases binding reagents may be attached to other moieties that can record information on a binding target barcode moiety, thereby forming an interaction moiety. FIGS. 4A and 4B illustrate enzymatic systems that record information on a binding target barcode moiety, thereby forming an interaction moiety. FIG. 4A depicts an analyte 105 and a co-localized binding target barcode moiety 101 attached to a solid support 100. A binding reagent comprising a particle 110 attached to an affinity reagent 111 and a ligase enzyme 420 is bound to the analyte 105. A ligase enzyme substrate comprising a ligation sequence 431 and a non-ligation sequence 430 is provided to the system. In the presence of the ligase enzyme 420, the ligation sequence 431 is attached to the binding target barcode moiety 101, thereby forming an interaction moiety. Enzymes such as sortase or subtiligase may be useful for forming interaction moieties. A binding target barcode moiety may be provided a recognition sequence that facilitates the enzymatic activity of a ligase enzyme. FIG. 4B depicts a similar system to that of FIG. 4A, however the binding reagent comprises an enzyme 421 such as terminal deoxynucleotidyl transferase (TdT) that extends a terminal end of the binding target barcode moiety 101. The resulting poly-T sequence at the end of the binding target barcode sequence of the binding target barcode moiety can be indicative of the binding of the binding reagent to the analyte 105.

In some cases, a binding interaction can be recorded by forming a separation tag that facilitates separation of only binding target barcode moieties that have been provided the separation tag. For example, a binding reagent containing a biotin ligase enzyme can attach a biotin molecule to a binding target barcode moiety, thereby facilitating separation of biotinylated barcode moieties from non-biotinylated barcode moieties using a separation medium containing an avidin-type protein. In another example, a poly-histidine tag can be ligated onto a binding target barcode moiety peptide strand by a binding reagent containing a ligase enzyme. Interaction moieties containing poly-histidine tags can be readily separated from barcode moieties without poly-histidine tags by a separation method such as antibody-based affinity chromatography or immobilized metal affinity chromatography (IMAC).

Methods of forming interaction moieties, such as those depicted in FIGS. 4A-4B, may be particularly useful for uniplex pluralities of binding reagents (i.e., pluralities of binding reagents, in which each binding reagent is attached to a same binding reagent identifier moiety). For characterizing multiplexed binding reagents, it may be preferable to provide each unique binding reagent with a unique binding reagent barcode moiety to distinguish interaction moieties formed by the binding interactions of each unique binding reagent.

A plurality of binding targets may be provided to a method or system set forth herein. In some cases, a plurality of binding reagents may be provided as an array of binding targets, in which the array may comprise a plurality of binding targets that are individually immobilized at discrete addresses of a solid support. Useful configurations of arrays of binding targets are described in U.S. Pat. Nos. 11,970,693 and 11,993,865, each of which is herein incorporated by reference in its entirety. An array of binding targets may be provided on a non-suspensible solid support. For example, an array may be formed on a surface of a fluidic cartridge or flow cell. Alternatively, a method or system set forth herein may utilize a plurality of binding targets that are provided in a fluid phase. For example, a plurality of binding targets may be attached to a bead or a plurality thereof, in which the bead of the plurality of beads is suspensible in a fluidic medium.

A plurality of binding targets may be provided to a method set forth herein, in which the plurality of binding targets is immobilized on a solid support. A solid support can comprise a plurality of sites, in which the plurality of binding targets is immobilized to the plurality of sites. In some configurations, only one binding target of the plurality of binding targets may be immobilized to a site of the plurality of sites. In other configurations, two or more binding targets of the plurality of binding targets can be immobilized to a site of the plurality of sites. Array compositions provided herein, such as array compositions formed by the deposition of binding targets utilizing anchoring moieties may be advantageous for a method set forth herein. An anchoring moiety may facilitate co-localization of a binding target with a binding target barcode moiety (e.g., the binding target and binding target barcode moiety are both attached to the anchoring moiety). A plurality of binding targets can be attached to a plurality of anchoring moieties, in which each site of the plurality of sites is attached to an anchoring moiety of the plurality of anchoring moieties. In some configurations, each anchoring moiety can be attached to only one binding target. In other configurations, each anchoring moiety can be attached to two or more binding targets.

In some configurations, a plurality of binding targets can be immobilized on a solid support, in which the solid support comprises a suspensible solid support (e.g., a bead, a particle, a microparticle, a nanoparticle, etc.). A plurality of binding targets immobilized on a suspensible solid support may be suspended, solvated, or otherwise mobile in a fluidic medium. In some configurations, a suspensible solid support may comprise a magnetic particle, an electrically-charged particle, or a sedimenting particle. In some configurations, a binding target may be attached to a suspensible solid support by an anchoring moiety.

In some configurations, each individual binding target of an array of binding targets may be separated on a solid support by an optically resolvable distance from any other binding target of the array of binding targets. In some cases, an array of binding targets may comprise a single-molecule array. Accordingly, a method of identifying a binding reagent from a library of binding reagents can comprise detecting binding of the binding reagent to a binding target of an array of binding targets at single-molecule resolution (e.g., detecting a signal from the binding reagent at a single address containing the binding target). An array of binding targets may comprise a plurality of addresses, each address containing one and only one immobilized binding target, in which the addresses have an average pitch as measured by the average separation between respective centerpoints of adjacent addresses.

Alternatively, an array of binding targets may be provided, in which binding targets of a plurality of binding targets are separated by an optically non-resolvable distance. An array of binding targets may comprise a plurality of addresses, in which each address comprises a plurality of binding targets (e.g., a protein microarray). An array address may comprise a plurality of binding targets, in which each binding target of the array address comprises the same primary amino acid structure. An array address may comprise a plurality of binding targets, in which each binding target of the array address comprises the same proteoform. An array address may comprise a plurality of binding targets, in which the array address comprises two or more binding targets having differing primary amino acid structures. An array address may comprise a plurality of binding targets, in which the array address comprises two or more binding targets having differing proteoforms.

An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

An array of binding reagents may be provided on a solid support or a surface thereof containing one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as binding sites for the coupling of binding targets or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method may comprise contacting a plurality of binding reagents to a plurality of binding targets. In some configurations, each binding reagent of the plurality of binding reagents has a same binding specificity. For example, a method may comprise contacting a plurality of first binding reagents to a plurality of binding targets, in which each binding reagent of the plurality of first binding reagents has a same binding specificity, then contacting a plurality of second binding reagents to a plurality of binding targets, in which each binding reagent of the plurality of second binding reagents has a same binding specificity, and in which the binding specificity differs between the first binding reagents and the second binding reagents. In some configurations, each binding reagent of the plurality of binding reagents has a differing binding specificity. For example, a method may comprise simultaneously contacting each binding reagent of a library of binding reagents to a plurality of binding targets.

After delivering a plurality of binding reagents to a plurality of binding targets and coupling a binding reagent of the plurality of binding reagents to the plurality of binding targets, a method may further comprise removing unbound binding reagents of the plurality of binding reagents from the plurality of binding targets. In some configurations, a plurality of binding reagents may be contacted to a plurality of binding targets in a first fluidic medium. A method may further comprise removing unbound binding reagents from the plurality of binding targets by contacting the plurality of binding targets with a second fluidic medium, thereby displacing the first fluidic medium containing the unbound binding reagents from contact with the plurality of binding targets. For an array of binding targets, displacing the first fluidic medium and/or unbound binding reagents can comprise rinsing the array of binding targets. The second fluidic medium may be substantially devoid of binding reagents. In a fluid phase, it may be useful to couple the binding targets to a solid phase (e.g., a magnetic bead, a sedimenting bead) that can be separated from the first fluidic medium (e.g., by magnetophoresis, by centrifugation or settling, etc.). In such a configuration, the first fluidic medium may be decanted or extracted from a vessel containing the binding targets. Unbound binding reagents may be removed from a plurality of binding targets before obtaining barcode moieties from bound binding reagents to inhibit the collection of barcode moieties from any binding reagent that did not actually bind to a binding target. In some cases, two or more rinsing media of differing compositions and/or stringencies (e.g., differing pHs, differing ionic strengths) may be contacted to a plurality of binding reagents, thereby facilitating removal of unbound, weakly bound, or non-specifically bound binding reagents.

A method of screening a library of binding reagents may comprise a step of obtaining a barcode moiety from a binding reagent bound to a binding target of a plurality of binding targets. Obtaining the barcode moiety may occur in the presence of the plurality of binding targets. Alternatively, obtaining the barcode moiety may occur in the absence of the plurality of binding targets. Accordingly, obtaining the barcode moiety in the absence of the plurality of binding targets can comprise one or more steps of: i) eluting the binding reagent from the plurality of binding targets, and ii) removing the binding reagent from the plurality of binding targets (e.g., rinsing the binding reagent from the plurality of binding targets in a fluidic medium).

In some configurations, obtaining a barcode moiety from a binding reagent can comprise separating the barcode moiety from the binding reagent. A barcode moiety may be separated from a binding reagent while the binding reagent is still bound to a binding target. A barcode moiety may be separated from a binding reagent after the binding reagent has been eluted from a binding target. A barcode moiety may be separated from a binding reagent after the binding reagent has been removed from contact with a plurality of binding targets. Separating a barcode moiety from a binding reagent may comprise enzymatically, chemically, or photolytically cleaving the barcode moiety from the binding reagent. Accordingly, a barcode moiety may be attached to a binding reagent by a labile linking group (e.g., a labile functional group, a protease-cleavable peptide, a nucleic acid containing a restriction site, a photocleavable functional group, etc.). A method may comprise contacting a binding reagent with a separating agent, wherein the separating agent is selected from an enzyme (e.g., a protease, a restriction enzyme), a chemical reagent, or a photon.

In some configurations, obtaining the barcode moiety from the binding reagent can comprise replicating the barcode moiety of the binding reagent. A barcode moiety may be replicated while the binding reagent is still bound to a binding target. A barcode moiety may be replicated after the binding reagent has been eluted from a binding target. A barcode moiety may be replicated after the binding reagent has been removed from contact with a plurality of binding targets. Replicating a barcode moiety may comprise forming a second coded moiety that contains information from the barcode moiety. If the barcode moiety comprises a nucleic acid, replicating the barcode moiety of the binding reagent can comprise performing a polymerase chain reaction with the barcode moiety. Accordingly, a method may comprise contacting the binding reagent with a polymerase enzyme and optionally a priming oligonucleotide. Alternatively, replicating a nucleic acid barcode moiety can comprise extending a double-stranded nucleic acid linking moiety that attaches a single-stranded barcode nucleic acid to a binding reagent. A method may further comprise separating the replicated barcode moiety from the binding reagent (e.g., via denaturation, via enzymatic, chemical, or photolytic cleavage, etc.). The skilled person will recognize that a replicated barcode moiety will be the reverse complement of a barcode moiety for a nucleic acid, so sequencing of the replicated barcode moiety will produce the inverse sequence of the barcode moiety. In some cases, performing a polymerase chain reaction to replicate a barcode moiety will further comprise amplifying the barcode moiety by the polymerase chain reaction, wherein amplifying the barcode moiety comprises forming a plurality of copies of the replicated barcode moiety.

A method may comprise a step of detecting a sequence of a barcode moiety. Detecting the sequence of the barcode moiety may further comprise transferring the barcode moiety to a sequencing device. In some configurations in which the barcode moiety comprises a nucleic acid, and the sequencing device can comprise a nucleic acid sequencing device. Accordingly, detecting the sequence of the barcode moiety may comprise detecting a nucleotide sequence of the barcode moiety. In other configurations in which the barcode moiety comprises a peptide, the sequencing device may comprise a peptide sequencing device. Accordingly, detecting the sequence of the barcode moiety can comprise detecting an amino acid sequence of the barcode moiety.

After a plurality of interaction moieties is detected, individual interaction moieties of the plurality of interaction moieties may be classified according to information contained within the individual interaction moieties, including: (i) information associated with a binding target, (ii) information associated with a binding reagent, or (iii) a combination thereof. Accordingly, interaction moieties of a plurality of interaction moieties can be quantified according to interaction moieties containing: (i) common information associated with a binding target, (ii) common information associated with a binding reagent, or (iii) common information associated with a same binding target and common information associated with a same binding reagent.

Classification and/or quantification of interaction moieties from detection data may be facilitated by a processor that is configured to analyze the detection data. Detection data may be transmitted from a detection device (e.g., a sequencing device) to a processor that is configured to classify and/or quantify the detection data. A processor may be configured to receive detection data from an interaction moiety and identify within the data one or more of: (i) information associated with a binding target, and (ii) information associated with a binding reagent. A processor may be further configured to, based upon classified or quantified detection data: (i) identify a binding reagent from a library of binding reagents that binds an epitope Q, and/or (ii) form a binding model, as set forth herein, for a binding reagent. A processor that classifies and/or quantifies detection data may utilize a training algorithm or a machine learning algorithm.

Methods and systems of the present disclosure may be especially useful for characterizing complex binding reagents. A complex binding reagent may comprise additional components in addition to an affinity reagent and one or more detectable labels (e.g., fluorophores). In some configurations, a binding reagent may comprise one or more additional components selected from: i) one or more additional affinity reagents, ii) one or more pendant polymer strands, and iii) a linking moiety that couples together two or more of the components of the binding reagent. A binding reagent may comprise a pendant polymer strand such as a synthetic polymer strand (e.g., polyethylene glycol), an oligonucleotide, or a peptide. In some configurations, a barcode moiety may be attached to a polymer strand of a binding reagent. In other configurations, a barcode moiety may not be attached to a polymer strand.

A binding reagent may comprise a linking moiety that attaches two or more components of a binding reagent together. A barcode moiety of a binding reagent of the library of binding reagents may be attached to a linking moiety of the binding reagent. In some configurations, a linking moiety may comprise a polymer strand or a particle. A particle may comprise an organic nanoparticle, an inorganic nanoparticle, a nucleic acid nanoparticle, a polymer particle, or a dendrimer.

A binding reagent of a library of binding reagents may be provided with one or more attached detectable labels (e.g., a fluorophore or a luminophore). A binding reagent may be provided with two or more attached detectable labels (e.g., at least about 2, 5, 10, 15, 20, 25, 30, 40, 50, or more than 50 detectable labels). It may be advantageous to provide a binding reagent comprising an attached detectable label if the binding reagent will be provided with the detectable label in an assay or other application. The presence of a fluorophore or luminophore may affect the binding characteristics of an affinity reagent, and can also affect the tendency of a binding reagent to form orthogonal binding interactions (i.e., non-specific binding). In some configurations, a method can include a step of detecting a signal from a fluorophore or luminophore at an address of an array of binding targets, thereby detecting the binding reagent. If an array is spatially-addressable, it may be possible to detect if a binding reagent is bound at an array address containing a binding target (i.e., likely bound specifically to the binding target), or bound at an array address that is devoid of a binding target (e.g., bound orthogonally to the array). In some configurations, a detectable label may be attached to a linking moiety of a binding reagent. In other configurations, a detectable label may be attached to an affinity reagent of a binding reagent.

Any of a variety of affinity reagents can be used in a composition or method set forth herein. Particularly useful affinity reagents include, but are not limited to, antibodies whether full length or functional fragments thereof (e.g., Fab′ fragments, F(ab′)₂ fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, Major Histocompatibility Complex (MHC) molecules, or functional fragments thereof. The exemplified affinity reagents can be used individually. Alternatively, an affinity reagent set forth herein can be used as a paratope or moiety of an affinity reagent having a plurality of paratopes or moieties. For example, an affinity reagent set forth herein can provide one paratope (or a subset of paratopes) of an affinity reagent having a plurality of paratopes. In some configurations, a composition or method set forth herein can lack one or more of the affinity reagents set forth herein.

### Screening of Binding Reagents

Provided herein are systems and methods for the screening of binding reagents, including the screening of binding reagents under equilibrium or non-equilibrium binding conditions. Screening of binding reagents may refer to the identification of one or more binding reagents having a particular binding characteristic (e.g., a binding specificity) from amongst a plurality of uncharacterized binding reagents. For example, a plurality of uncharacterized binding reagents may be screened to identify a binding reagent that binds to a specific protein or epitope of the specific protein. In another example, a plurality of uncharacterized binding reagents may be screened to identify a binding reagent that binds to a set of proteins, or an epitope common to each protein of the set of proteins.

Methods and system provided herein may be useful for selecting a binding reagent with a desired binding specificity and/or affinity from a library of binding reagents. A library of binding reagents may comprise a population of binding reagents that contain diversity with respect to the binding specificity and/or affinity amongst members of the library of binding reagents. The diversity with respect to binding specificity and/or affinity amongst members of the library of binding reagents may arise due to a diversity of structures of analyte, or due to epitope-binding regions of the binding reagents. For example, binding reagents comprising antibody affinity reagents may differ with respect to binding specificity and/or affinity due to differences in amino acid sequence of complementarity-determining regions (CDRs) of the respective antibodies. In another example, binding reagents comprising aptamer affinity reagents may differ with respect to binding specificity and/or affinity due to differences in nucleotide sequence of analyte- or epitope-binding regions of the respective aptamers.

A library of binding reagents may comprise a plurality of unique binding reagents, as determined by binding specificity and/or affinity of each respective unique binding reagent. A library of binding reagents may comprise a plurality of unique binding reagents, as determined by sequence or structure of an analyte- or epitope-binding region of each respective unique binding reagent. A library of binding reagents may comprise two or more binding reagents that are degenerate with respect to binding specificity (i.e., two structurally differing binding reagents that bind a same binding target or epitope thereof). A library of binding reagents may comprise a naïve library of affinity reagents (e.g., a library containing two or more unique binding reagents with unknown binding affinities for a binding target). A library of binding reagents may comprise affinity reagents produced by a targeted maturation campaign of a characterized affinity reagent (e.g., a population of binding reagents, each binding reagent containing a structural variant of a single affinity reagent). A library of affinity reagents can comprise a population of affinity reagents, each affinity reagent differing due to substitution of one or more residues (e.g., amino acids, nucleotides) with naturally-occurring, non-natural, or modified residues.

A library of binding reagents may comprise at least about 2, 5, 10, 50, 100, 250, 500, 1000, 5000, 10000, 50000, 10⁵, 10⁶, 10⁷, 10⁸, 10⁹, or more than 10⁹, unique binding reagents (e.g., as determined by differences in chemical structure of a complementarity-determining region). Alternatively or additionally, a library of binding reagents may comprise no more than about 10⁹, 10⁸, 10⁷, 10⁶, 10⁵, 50000, 10000, 5000, 1000, 500, 250, 100, 50, 10, 5, or less than about 5 unique binding reagents.

A plurality of binding reagents may be screened to identify a binding reagent that is useful for an analyte characterization assay. Binding reagents may be useful for certain analyte characterization assays, including identification of peptides or proteins, and sequencing of peptides or proteins. Example of some analyte characterization assays are described below in the section titled “Single-Analyte Assays.”

Methods and system provided herein may be useful for screening a plurality of uncharacterized binding reagents to identify a binding reagent that recognizes and/or binds to an epitope of length n (where n is about 2, 3, 4, 5, 6, 7, 8, 9, 10, 15, 20, 25, 30, or more than 30 amino acids) having a sequence of X₁X₂ . . . . Xₙ (e.g., X₁X₂X₃ for n=3, X₁X₂X₃X₄ for n=4, etc.), where each X represents any possible amino acid, including the 20 naturally occurring amino acids, and optionally including non-natural or modified amino acids. In some cases, methods and systems provided herein may be useful for screening a plurality of uncharacterized binding reagents to identify a binding reagent that recognizes and/or binds to an epitope of length n having a sequence of X₁X₂ . . . . Xₙ, in which the binding reagent binds to the epitope in at least about 10% (e.g., at least about 20%, 30%, 40%, 50%, 60%, 70%, 80%, 90%, 95%, or more than 95%) of sequence contexts αX₁X₂ . . . . Xₙβ, wherein α and β are flanking sequences of epitope X₁X₂ . . . . Xₙ. Flanking sequences α and/or β may comprise at least one amino acid (e.g., at least 2 amino acids, 3 amino acids, 4 amino acids, etc), including any of the 20 naturally occurring amino acids, and optionally including non-natural or modified amino acids (e.g., amino acids with sidechains modified to include biological post-translation modifications such as phosphorylation, methylation, etc.; amino acids with sidechains modified to include synthetic modifications such as click-type reagents, PEGylation, etc.). Flanking sequences a and/or β may comprise a moiety other than an amino acid (e.g., a PEG moiety, a nucleic acid, etc.).

Methods and system provided herein may be useful for screening a plurality of uncharacterized binding reagents to identify a binding reagent that recognizes and/or binds to an epitope of length n having a sequence of X₁X₂ . . . . Xₙ, where at least one amino acid of the sequence X₁X₂ . . . . Xₙ is modified. In some cases, the modification to the amino acid may comprise a naturally-occurring modification (e.g., a post-translational modification that occurs in a biological system) or a synthetic modification.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

In some cases, methods and systems provided herein may be useful for screening a plurality of uncharacterized binding reagents to identify a binding reagent that recognizes and/or binds to an epitope of length n having a sequence of X₁X₂ . . . . Xₙ, in which the binding reagent binds to the epitope in at least about 10% (e.g., at least about 20%, 30%, 40%, 50%, 60%, 70%, 80%, 90%, 95%, or more than 95%) of sequence contexts αX₁X₂ . . . . Xₙβ, wherein a and β are flanking sequences of epitope X₁X₂ . . . . Xₙ, and where at least one amino acid of the flanking sequence a and/or B is modified. In some cases, the modification to the amino acid may comprise a naturally-occurring modification (e.g., a post-translational modification found in a biological system, such as phosphorylation or methylation) or a synthetic modification (e.g., modification to form a click-type reagent).

Methods and system provided herein may be useful for screening a plurality of uncharacterized binding reagents to identify a binding reagent that recognizes and/or binds to a terminal amino acid, a modified terminal amino acid, a terminal epitope, or a modified terminal epitope. Certain methods of peptide sequencing may utilize sequential or serial modification and cleavage of one or more terminal amino acids, for example via an Edman-type degradation process. Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA; DOI: 10.1007/978-1-4899-1031-8_8). Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected. Methods and system set forth herein may be useful for screening a plurality of binding reagents to identify a binding reagent that recognizes and/or binds to any modified form of a terminal amino acid before the amino acid is cleaved from a peptide. Methods and system set forth herein may be useful for screening a plurality of binding reagents to identify a binding reagent that recognizes and/or binds to an epitope containing a modified form of a terminal amino acid before the amino acid is cleaved from a peptide.

In an aspect, provided herein is a method of selecting a binding reagent, comprising: a) contacting a library of binding reagents to a plurality of binding targets, wherein each binding target of the plurality of binding targets comprises an epitope Θ, wherein each binding reagent of the library of binding reagents contains a unique binding specificity, and wherein each binding reagent of the library of binding reagents comprises: i) an affinity reagent having the unique binding specificity, ii) a barcode moiety that is specific to the affinity reagent, and iii) a linking moiety that attaches the affinity reagent to the barcode moiety, b) coupling a binding reagent of the library of binding reagents to a binding target of the plurality of binding targets, c) obtaining the barcode moiety from the binding reagent coupled to the peptide target, d) detecting a sequence of the barcode moiety, and e) based upon the sequence of the barcode moiety, determining the structure of the affinity reagent.

In another aspect, provided herein is a method of selecting a binding reagent having a binding specificity for an epitope Θ, comprising: (a) providing a plurality of binding targets, wherein each binding target of the plurality of binding targets comprises the epitope Θ, and wherein each binding target of the plurality of binding targets is co-localized with a unique binding target barcode moiety, (b) providing a library of binding reagents wherein each binding reagent of the library of binding reagents differs from each other binding reagent of the library of binding reagents with respect to binding specificity, and wherein each binding reagent of the library of binding reagents is attached to a unique binding reagent barcode moiety, (c) combining the plurality of binding targets with the library of binding reagents, thereby coupling binding reagents of the library of binding reagents to binding targets of the plurality of binding targets, (d) forming a plurality of interaction moieties, wherein each interaction moiety of the plurality of interaction moieties comprises information from a binding target barcode moiety of a binding target of the plurality of binding targets and information from the binding reagent barcode moiety, and (e) detecting two or more interaction moieties containing information from a same binding reagent barcode moiety, thereby selecting the binding reagent of the library of binding reagents having a binding specificity for the epitope Θ.

A method of selecting a binding reagent may include multiple screening steps. For example, a second screening step utilizing a reduced library of affinity reagents (e.g., as selected by highest observed sequences counts during an initial screen) may be performed against a same plurality of binding targets. In another example, a library of affinity reagents (e.g., a full library of affinity reagents, a reduced library of affinity reagents) may be screened against a second plurality of binding targets, in which the second plurality of binding targets differ from a first plurality of binding targets utilized in a first screening step.

In some configurations, a plurality of binding targets may be substantially homogeneous with respect to the structure of binding targets of the plurality of binding targets. For example, a plurality of binding targets can comprise a plurality of binding targets, each binding target having an identical primary amino acid sequence. In another example, a plurality of binding targets can comprise a plurality of binding targets, each binding target containing an identical proteoform of a single protein. It may be useful to provide a plurality of binding targets that is substantially homogeneous with respect to the structure of binding targets of the plurality of binding targets when screening and selecting for a binding reagent that is specific to a single protein.

Alternatively, in some configurations, a plurality of binding targets may be heterogeneous with respect to the structure of binding targets of the plurality of binding targets. In some configurations, a plurality of binding targets may be provided with a plurality of unique binding targets, as determined by primary amino acid sequence, in which each binding target of the plurality of binding targets comprises an epitope Θ of length n (where n equals at least about 2, 3, 4, 5, 6, 7, 8, 9, 10, 15, 20, 25, 30 or more than 30 amino acids), in which the epitope Θ has a structure X₁X₂ . . . . Xₙ, where each X can be independently selected from any naturally-occurring, non-natural, or modified amino acid (e.g., each peptide of a plurality of binding target peptides contains an epitope having the amino acid sequence DTR).

In particular configurations, a plurality of binding targets may be provided, in which the plurality of binding targets contains at least about 10, 20, 50, 100, 200, 400, 1000, 2000, 5000, 10000, 15000, 20000, 50000, 100000, 1000000, or more than 1000000 sequence contexts of an epitope Θ of length n (where n equals at least about 2, 3, 4, 5, 6, 7, 8, 9, 10, 15, 20, 25, 30 or more than 30 amino acids), in which the epitope Θ has a structure X₁X₂ . . . . Xₙ, where each X can be independently selected from any naturally-occurring, non-natural, or modified amino acid (e.g., each peptide of a plurality of binding target peptides contains an epitope having the amino acid sequence DTR). Each sequence context may comprise a structure αΘβ, in which a and B are flanking amino acid sequences of epitope Θ, in which α and β can independently comprise about 0, 1, 2, 3, or more than 3 amino acids, and in which α and β can contain any naturally-occurring, non-natural, or modified amino acid. For example, in a particular configuration, a plurality of binding targets may comprise at least 400 sequence contexts of epitope Θ, in which α and β are each a single amino acid, and in which every permutation of α and β for the 20 naturally-occurring amino acids is present in the at least 400 sequence contexts (e.g., α=A, β=A; α=A, β=C; α=A, β=D, etc.).

In some configurations, a plurality of binding targets may be provided with a plurality of unique binding targets, as determined by primary amino acid sequence, in which each binding target of the plurality of binding targets comprises an epitope Θ of length n (where n equals at least about 2, 3, 4, 5, 6, 7, 8, 9, 10, 15, 20, 25, 30, or more than 30 amino acids), in which the epitope Θ has a structure X₁X₂ . . . . Xₙ, where each X can be independently selected from any naturally-occurring, non-natural, or modified amino acid, and in which at least one residue X of epitope Θ contains a modified structure (e.g., a post-translational modification, a non-natural amino acid). For example, a plurality of binding targets can comprise a plurality of binding targets, in which each binding target of the plurality of binding targets comprises an epitope Θ, in which Θ has a structure DC*R, in which C* can be any post-translational modification of the amino acid cysteine.

In particular cases, a plurality of binding targets may be provided, in which the plurality of binding targets contains at least about 10, 20, 50, 100, 200, 400, 1000, 2000, 5000, 10000, 15000, 20000, 50000, 100000, 1000000, or more than 1000000 sequence contexts of an epitope Θ of length n (where n equals at least about 2, 3, 4, 5, 6, 7, 8, 9, 10, 15, 20, 25, 30, or more than 30 amino acids), in which the epitope Θ has a structure X₁X₂ . . . . Xₙ, where each X can be independently selected from any naturally-occurring, non-natural, or modified amino acid. Each sequence context may comprise a structure αΘβ, in which α and β are flanking amino acid sequences of epitope Θ, in which α and β can independently comprise about 0, 1, 2, 3, or more than 3 amino acids, and in which at least one of α and β contains a non-natural, or modified amino acid. For example, in a particular configuration, a plurality of binding targets may comprise at least 50 sequence contexts of epitope Θ, in which α comprises any post-translational modification of amino acid cysteine, and in which β can comprise any naturally-occurring, non-natural, or modified amino acid.

In some configurations, a plurality of binding targets may be provided with a plurality of unique binding targets, as determined by primary amino acid sequence, in which each binding target of the plurality of binding targets comprises a same terminal modified amino acid (e.g., modified for cleavage by an Edman-type degradation reaction). For example, a plurality of binding targets may be provided, in which each binding target comprises a cyclical phenylthiocarbamoyl Edman complex derivative at its N-terminus. In some configurations, a plurality of binding targets may be provided with a plurality of unique binding targets, in which each binding target of the plurality of binding targets comprises a same terminal modified amino acid (e.g., modified for cleavage by an Edman-type degradation reaction) in a different sequence context. For example, a plurality of binding targets may comprise at least about 10, 50, 100, 200, 400, 1000, 2000, 5000, 10000, 15000, 20000, 50000, 100000, 1000000, or more than 1000000 sequence contexts of structure X₁X₂*, where X₁ is any naturally-occurring, non-natural, or modified amino acid, and where X₂* is a cyclical phenylthiocarbamoyl Edman complex derivative of any naturally-occurring, non-natural, or modified amino acid.

In some cases, a binding target of a plurality of binding targets may comprise an amino acid sequence of a known protein (e.g., a partial amino acid sequence). In particular cases, a binding target of a plurality of binding targets may comprise a complete amino acid sequence of a known protein. In some configurations, a binding target of a plurality of binding targets may comprise a repeat of an epitope Θ (e.g., a peptide comprising a sequence ΘΘΘ, a peptide comprising a sequence ΘXΘXΘ, where X is a spacing moiety that can comprise amino acids and/or a polymer linker). In some configurations, a plurality of binding targets can comprise a plurality of binding targets, in which each binding target of the plurality of binding targets comprises an identical amino acid sequence. Alternatively, a plurality of binding targets can comprise a plurality of binding targets, in which the plurality of binding targets comprises two or more binding targets that differ with respect to amino acid sequence.

A plurality of binding targets may comprise a plurality of peptides, in which a peptide of the plurality of peptides is at least about 5, 10, 15, 20, 25, 30, 40, 50, 100, or more than 100 amino acids in length. Alternatively or additionally, an array of binding targets may comprise a plurality of peptides, in which a peptide of the plurality of peptides is no more than about 100, 50, 40, 30, 25, 20, 15, 10, 5, or less than 5 amino acids in length. A plurality of binding targets may comprise a plurality of peptides, in which each peptide is at least about 5, 10, 15, 20, 25, 30, 40, 50, 100, or more than 100 amino acids in length. Alternatively or additionally, a plurality of binding targets may comprise a plurality of peptides, in which each peptide is no more than about 100, 50, 40, 30, 25, 20, 15, 10, 5, or less than 5 amino acids in length.

Novel affinity reagents that can be incorporated into binding reagents may be generated by any method known in the art. Methods of developing affinity reagents can include Systematic evolution of ligands by exponential enrichment (SELEX), phage display, yeast display, mammalian cell display, insect cell display, ribosome display, particle display, peptimer evolution, peptimer design, and inoculation. In some examples, affinity reagents may be designed using structure based design methods.

A method may comprise contacting a library of binding reagents to a plurality of binding targets. The library of binding reagents may be contacted to the plurality of binding targets simultaneously or iteratively. Simultaneous contact of a library of binding reagents can comprise contacting each unique binding reagent of the library of binding reagents to a plurality of binding reagents at the same time. Iterative contact of a library of binding reagents can comprise multiple contacting steps, with each contacting step comprising contacting a single unique binding reagent of the library of binding reagents to a plurality of binding reagents. Alternatively, iterative contact of a library of binding reagents can comprise multiple contacting steps, with each contacting step comprising contacting a subset of unique binding reagents of the library of binding reagents to a plurality of binding reagents. It shall be understood that a method step comprising contacting a unique binding reagent to a plurality of binding targets can refer to contacting a plurality of the unique binding reagent to the plurality of binding targets provided that each member of the plurality of the unique binding reagent has the same structure and/or binding specificity (i.e., each binding reagent contains structural identical affinity reagents).

Table I depicts a multi-cycle method for contacting a library of 6 unique binding reagents to a plurality of binding targets. In cycles 1 through 6, a single unique binding reagent of the 6 unique binding reagents is contacted to the plurality of binding targets for each respective cycle. In cycle 7, all 6 unique binding reagents are simultaneously contacted to the plurality of binding targets. In cycles 8 and 9, non-overlapping subsets of unique binding reagents are contacted to the plurality of binding targets. Collectively, cycles 8 through 12 comprise contacting differing overlapping subsets of unique binding reagents to the plurality of binding targets. Cycles 13 through 18 include repeating cycles 7, 3, 4, 10, 11, and 9, respectively. Use of both overlapping and non-overlapping sets or subsets of binding reagents, as well as repeating certain cycles, may facilitate identification of a higher affinity or more avid binding reagent. For example, in the process of Table I, if cycles 3 and 4 result in the identification of binders for a binding target, then cycles 7 through 18 will confirm their binding specificity for the binding target and potentially facilitate identification of whether unique binding reagent 3 or 4 more strongly competes for the binding target when both are contacted simultaneously (cycles 7, 10, 11, 13, 17, and 18).

TABLE I

| Unique | | | | | | |
|---|---|---|---|---|---|---|
| Binding | Cycle Number | | | | | |
| Reagent | 1 | 2 | 3 | 4 | 5 | 6 |
| 1 | X | | | | | |
| 2 | | X | | | | |
| 3 | | | X | | | |
| 4 | | | | X | | |
| 5 | | | | | X | |
| 6 | | | | | | X |
| Unique | | | | | | |
| Binding | Cycle Number | | | | | |
| Reagent | 7 | 8 | 9 | 10 | 11 | 12 |
| 1 | X | X | | | | X |
| 2 | X | X | | X | | |
| 3 | X | X | | X | X | X |
| 4 | X | | X | X | X | |
| 5 | X | | X | | X | X |
| 6 | X | | X | | | |
| Unique | | | | | | |
| Binding | Cycle Number | | | | | |
| Reagent | 13 | 14 | 15 | 16 | 17 | 18 |
| 1 | X | | | X | | |
| 2 | X | | | X | | |
| 3 | X | X | | X | X | |
| 4 | X | | X | | X | X |
| 5 | X | | | | X | X |
| 6 | X | | | | | X |

In some cases, contacting the library of binding reagents to the plurality of binding targets can comprise contacting a first binding reagent of the library of binding reagents to the plurality of binding targets. The method may further comprise determining a presence or absence of a barcode moiety from the first binding reagent of the library of binding reagents. After determining a presence or absence of a binding interaction between the first binding reagent and a binding target of the plurality of binding targets, other members of the library of binding targets may be assessed sequentially. Accordingly, a method may further comprise: i) repeating the contacting and coupling steps with a second binding reagent of the library of binding reagents, and ii) determining a presence or absence of a barcode moiety from the second binding reagent of the library of binding reagents. A method may further comprise, for a library of N binding reagents, repeating steps i) and ii) for a third binding reagent through an Nth binding reagent.

A method of screening a library of binding reagents may lead to the identification of more than one binding reagent that can bind to a binding target of an array of binding targets. Accordingly, a method may comprise obtaining and/or detecting two or more barcode moieties from the two or more identified binding reagents. It may be useful to provide a library of binding reagents, in which two or more identical copies of each unique binding reagent is provided in the library. Quantification of the total number of bound binding reagents for each unique binding reagent of a library of binding reagents may be useful for determining the most likely, strongest, or most avid binder from a library of binding reagents for a binding target. Accordingly, a method may comprise obtaining a plurality of barcode moieties from each of two or more binding reagents of the library of binding reagents. A method may further comprise determining for each of the two or more binding reagents a quantity of the plurality of barcode moieties. A method may further comprise determining the structure of the affinity reagent for the binding reagent having the largest quantity of detected barcode moieties.

After a binding reagent that binds to a particular binding target has been selected, it may be preferable to identify the structural features of the binding reagent that give rise to the binding specificity for the binding target. Specifically, it may be preferable to identify the structural features of an affinity reagent of a binding target that gives rise to the binding specificity of the binding reagent for the binding target. The use of differing barcodes to differentiate differing binding reagents or differing affinity reagents may be useful for correlating affinity reagent structure to the binding characteristics of a particular binding reagent. A method of selecting a binding reagent from a library of binding reagents may comprise determining a structure of the affinity reagent. For example, if a binding reagent comprises an antibody or an antibody fragment, a method may comprise a step of determining a structure or amino acid sequence of a paratope of the antibody or antibody fragment. In another example, if a binding reagent comprises an aptamer or a mini-protein binder, a method may comprise a step of determining a residue sequence of the aptamer or mini-protein binder.

In some configurations, the structure of an affinity reagent of each binding reagent present in a library of binding reagents may be determined before a method of screening set forth herein. Such an approach may be practical, for example, in affinity reagent maturation campaigns, in which variants of an affinity reagent of known structure are produced to provide a library of variants of a binding reagent. Subsequently, the library of variants of the binding reagent may be screened to identify a variant with enhanced binding characteristics. Accordingly, before selecting a binding reagent from a library of binding reagents, barcode moieties may be provided to each structurally unique binding reagent of the library of binding reagents, in which the barcode moiety of each binding reagent corresponds to the structure and/or binding specificity of the affinity reagent of the binding reagent. Each binding reagent containing an affinity reagent with a structurally-identical binding region may be provided a copy of the same barcode moiety. In some configurations, a barcode moiety may comprise a code or sequence (e.g., a nucleotide sequence, an amino acid sequence) that corresponds to the structure of the affinity reagent of the binding reagent to which the barcode moiety is attached. In some configurations, a barcode moiety may comprise a code or sequence (e.g., a nucleotide sequence, an amino acid sequence) that encodes the actual structure of the affinity reagent of the binding reagent to which the barcode moiety is attached. For example, a binding reagent containing an antibody may comprise a barcode moiety containing a nucleotide sequence or an amino acid sequence of the complementarity-determining region (CDR) of the antibody.

In some configurations, the structure of an affinity reagent of each binding reagent present in a library of binding reagents may be determined after a method of screening set forth herein. Barcode moieties may be useful for capturing selected moieties. For example, a solid support containing a complementary barcode moiety may bind the barcode moiety of a binding reagent eluted from an array of binding targets. A captured binding reagent may be subsequently sequenced to determine its structure. Identification of the barcode moiety of a binding reagent may also facilitate separation of like affinity reagents from a plurality of affinity reagents containing multiple copies of the binding reagent. It may be preferable to obtain multiple copies of the selected binding reagent so that multiple copies of the affinity reagent of the selected binding reagent can be provided to a sequencing device. Methods for determining the structure of an affinity reagent are known in the art, and any suitable method may be used. Aptamers and mini-protein binders can be sequenced via nucleic acid or peptide sequencing devices, respectively. Numerous approaches exist for antibody sequencing, including tandem mass spectrometry and Edman degradation methods.

In some cases, a method of selecting a binding reagent for an epitope Θ may comprise: (i) identifying a first set of interaction moieties, wherein each interaction moiety of the first set of interaction moieties comprises information from a first same binding reagent barcode moiety of a first binding reagent of the library of binding reagents; (ii) identifying a second set of interaction moieties, wherein each interaction moiety of the second set of interaction moieties comprises information from a second same binding reagent barcode moiety of a second binding reagent of the library of binding reagents; and (iii) determining which of the first set of interaction moieties and the second set of interaction moieties contains a greater quantity of interaction moieties, thereby selecting the binding reagent of the library of binding reagents having a binding specificity for the epitope Θ.

In some cases, a method of selecting a binding reagent for an epitope Θ may comprise identifying a binding reagent that binds to binding targets containing the epitope Θ in a variety of sequence contexts, as set forth herein. In some cases, a binding reagent may be selected from a library of binding reagents if at least one binding interaction is observed (e.g., via the detection of an interaction moiety) between the binding reagent and at least about 5% (e.g., at least about 10%, 15%, 20%, 30%, 40%, 50%, or more than 50%) of all sequence contexts contained amongst a plurality of binding targets. In some cases, a binding reagents may be selected from a library of binding reagents if a plurality of binding interactions is observed (e.g., via the detection of an interaction moiety) between the binding reagent and each individual binding target of a set of binding targets, in which the set of binding targets contains at least about 5% (e.g., at least about 10%, 15%, 20%, 30%, 40%, 50%, or more than 50%) of all sequence contexts contained amongst a plurality of binding targets.

### Characterization of Binding Reagents

Methods and systems set forth herein may also be useful for characterizing binding reagents, such as binding reagents selected by a screening method set forth herein. Characterization of a binding reagent can refer to the determination of binding characteristics of a single binding reagent against a plurality of differing binding targets. Characterization of a binding reagent can include confirming the primary, superordinate, or on-target binding specificity of the binding reagent (i.e., confirming the binding specificity of the binding reagent for a target that the binding reagent was selected against). For example, characterizing a binding reagent that was selected to bind to an epitope DTR can comprise detecting binding of the binding reagent to binding targets containing the epitope DTR. Characterization of a binding reagent can further include determining one or more secondary, subordinate, or off-target binding interactions (e.g., binding of the binding reagent to binding targets other than binding targets against which the binding reagent was selected). For example, characterizing a binding reagent that was selected to bind to an epitope DTR can comprise detecting binding of the binding reagent to binding targets containing similar amino acid sequences (e.g., DTE, DTK, DTW, etc.). In another example, characterizing a binding reagent that was selected to bind to an epitope DTR can comprise detecting binding of the binding reagent to binding targets containing non-similar amino acid sequences (e.g., HSH, HGK, ARH, etc.). Characterization of a binding reagent can further include determining one or more orthogonal binding interactions (e.g., binding of the binding reagent to objects other than binding targets).

Characterization of a binding reagent can also include measurement of a binding affinity for the binding reagent. Measurement of binding affinity for a binding reagent can include quantitative measurements of binding characteristics such as dissociation constant (K_{D}), association rate constant (kₒₙ), and dissociation rate constant (k_{off}). For a binding reagent that is observed to form on-target and off-target binding interactions, characterization of the binding reagent may include measuring binding affinities for on-target binding interactions and off-target binding interactions. Certain methods of analyte characterization set forth herein may utilize probabilistic models of binding interactions to analyze measurement outcomes of observed binding interactions between binding reagents and analytes. Accordingly, characterization of a binding reagent may include: i) observing presence or absence of binding of the binding reagent to a plurality of differing binding targets, and ii) based upon the observed presence or absence of binding of the binding reagent to the plurality of differing binding targets, determining a probabilistic model of binding for the binding reagent to the differing binding targets.

Compositions comprising a plurality of binding targets, as set forth herein, may be useful for characterizing a binding reagent. In some configurations, a method or system may utilize an array of binding targets comprising a plurality of immobilized peptide binding targets. In some configurations, a method or system may utilize an array of binding targets comprising a plurality of immobilized protein binding targets. In other configurations, a method or system may utilize a plurality of binding targets provided in a fluid phase. In some cases, a method of selecting a binding reagent from a library of binding reagents with a plurality of binding targets may further comprise characterizing the selected binding reagent with the plurality of binding targets. In some cases, a method of selecting a binding reagent from a library of binding reagents with a first plurality of binding targets may further comprise characterizing the selected binding reagent with a second plurality of binding targets.

Useful configurations of pluralities of binding targets are described in the section titled “Screening of Binding Reagents.” Some described compositions contain pluralities of binding targets, in which each binding target comprises a structural feature that was common to all of the binding targets, such as a common epitope Q, or a common modified terminal amino acid. In some cases, the plurality of binding targets comprises a plurality of sequence contexts, in which the sequence contexts comprise one or more flanking structures adjacent to the common structural feature. For example, a plurality of binding targets can comprise a common epitope Θ, in which the plurality of binding targets comprises a plurality of sequence contexts of Θ with structure αΘβ, where α and β are flanking sequences of at least one amino acid selected from any naturally-occurring, non-natural, or modified amino acid.

For a binding reagent selected by screening against a specific binding target, such as a common epitope (or a common modified terminal amino acid, it may be preferable to provide a plurality of binding targets comprising a first set of binding targets containing the specific binding target, and a second set of binding targets that do not comprise the specific binding target. The presence of binding targets that do not comprise the specific binding target against which the binding reagent was screened may facilitate characterization of the binding specificity and/or binding affinity of the binding reagent for off-target binding targets.

In some configurations, a method or system for characterizing a binding reagent may utilize a plurality of binding targets, in which the binding targets of the plurality of binding targets each independently comprise an epitope of an epitope set ψ, in which each epitope of the epitope set ψ is at least about 2, 3, 4, 5, 6, 7, 8, 9, 10, 15, 20, 25, 30, or more than 30 amino acids in length, and in which each epitope of epitope set ψ independently has a structure X₁X₂ . . . . Xₙ, where each X is independently selected from any naturally-occurring, non-natural, or modified amino acid. For example, for the 20 naturally-occurring amino acids, the epitope set ψ could contain a complete permutation of all possible amino acid combinations, thereby providing 20″ possible epitopes of length n (e.g., for n=2, 400 possible unique epitopes, for n=3, 8000 possible unique epitopes, etc.). In another example, the epitope set ψ could contain at least about 5% (e.g., at least about 10%, 20%, 30%, 40%, 50%, 60%, 70%, 80%, 90%, 95%, 99%, 99.9%, or more than 99.9%) of all possible amino acid combinations for a set of amino acids. In another example, the epitope set ψ could contain no more than about 99.9% (e.g., no more than about 99%, 95%, 90%, 80%, 70%, 60%, 50%, 40%, 30%, 20%, 10%, 5%, or less than 5%) of all possible amino acid combinations for a set of amino acids.

In some configurations, a plurality of binding targets may each independently comprise an epitope of an epitope set ψ, in which each epitope of the epitope set ψ is provided in a plurality of sequence contexts, as set forth herein. In some configurations, a plurality of binding targets may comprise a plurality of binding targets, in which the binding targets of the plurality of immobilized binding targets each independently comprise an epitope of an epitope set ψ, in which each epitope of the epitope set ψ is provided in an identical sequence context. In some configurations, a plurality of binding targets may comprise a plurality of binding targets, in which the binding targets of the plurality of immobilized binding targets each independently comprise an epitope of an epitope set ψ, in which each epitope of the epitope set ψ is flanked by a spacing moiety that is substantially devoid of amino acids. In some configurations, a plurality of binding targets may each independently comprise an epitope of an epitope set ψ, in which each epitope of the epitope set ψ is provided two or more times (e.g., a peptide comprising a peptide comprising ΘΘΘ, wherein Θ is an epitope of ψ; a peptide comprising a peptide comprising ΘφΘηΘ, wherein Θ is an epitope of ψ and where φ and η are spacing moieties or flanking amino acid sequences, etc.).

In some configurations, a method or system for characterizing a binding reagent may utilize a plurality of binding targets comprising a plurality of proteins. A plurality of proteins may comprise one or more polypeptides having a primary amino acid sequence of a protein found in a naturally-occurring or engineered biological system. A plurality of proteins may comprise one or more polypeptides derived from a naturally-occurring or engineered biological system. A plurality of proteins may comprise one or more polypeptides obtained from a naturally-occurring or engineered biological system. A protein of a plurality of proteins may be produced transgenically or synthetically. A plurality of binding targets may comprise a polypeptide that is synthesized in situ, in which the polypeptide has a full or partial primary amino acid sequence of a naturally-occurring protein. A plurality of binding targets may comprise a plurality of peptide fragments, in which the plurality of peptide fragments comprises partial amino acid sequences of the primary amino acid sequences of one or more naturally-occurring proteins. A plurality of binding targets may comprise a plurality of peptide fragments, in which each peptide fragment of the plurality of peptide fragments comprises a modified terminal amino acid, as set forth herein.

A plurality of binding targets may comprise one or more polypeptides having a proteoform of a protein found in a naturally-occurring or engineered biological system. A proteoform can include a splice isoform of a protein, a protein with at least one post-translationally modified amino acid residue, a protein with at least one synthetically modified amino acid residue, or a combination thereof. A plurality of binding targets may comprise a plurality of proteins, in which one or more proteins of the plurality of proteins comprise a proteoform of a protein found in a naturally-occurring or engineered biological system. A plurality of binding targets may comprise a plurality of proteins, in which each protein of the plurality of proteins comprises a proteoform of the protein found in a naturally-occurring or engineered biological system. A plurality of binding targets may comprise a plurality of proteins, in which proteins of a first set of proteins of the plurality of proteins each individually comprise a proteoform of the protein found in a naturally-occurring or engineered biological system, and in which proteins of a second set of proteins of the plurality of proteins each individually do not comprise a proteoform of the protein found in a naturally-occurring or engineered biological system. A plurality of binding targets may comprise a protein having a full-length primary amino acid sequence, for example as determined by a gene encoding the protein amino acid sequence. A plurality of binding targets may comprise a protein having a truncated primary amino acid sequence, such as a splice isoform or a degradation product of the protein.

A method or system for characterizing a binding reagent that is selected to bind to an epitope Θ may utilize a plurality of binding targets, wherein the plurality of binding targets comprises a plurality of proteins comprising the epitope Θ. A plurality of binding targets can comprise a plurality of unique proteins (i.e., each protein of the plurality of unique proteins having a unique primary amino acid sequence from each other protein of the plurality of unique proteins), in which each protein of the plurality of unique proteins comprises a common epitope Θ. A plurality of binding targets can comprise a plurality of unique proteins (i.e., each protein of the plurality of unique proteins having a unique proteoform from each other protein of the plurality of unique proteins), in which each protein of the plurality of unique proteins comprises a common epitope Θ. A plurality of binding targets can comprise a plurality of unique proteins (i.e., each protein of the plurality of unique proteins having a unique proteoform from each other protein of the plurality of unique proteins), in which each protein of the plurality of unique proteins comprises a common epitope Θ containing a modified form of an amino acid residue of the common epitope Θ. A plurality of binding targets can comprise a plurality of unique proteins (i.e., each protein of the plurality of unique proteins having a unique proteoform from each other protein of the plurality of unique proteins), in which each protein of the plurality of unique proteins comprises a common epitope Θ containing an identical modified form of an amino acid residue of the common epitope Θ.

A plurality of binding targets may be provided to a method of binding reagent characterization for the purpose of forming a binding profile of the binding reagent. A binding profile may provide a qualitative and/or quantitative description of the binding behavior of a binding reagent with one or more binding targets. Information contained in a binding profile of an affinity reagent may be empirically derived, theoretically predicted, statistically modeled, or a combination thereof.

In some cases, it may be preferable to provide a plurality of binding targets that contains each binding target provided in a binding profile. For example, a particular protein (as defined by the protein's full-length primary amino acid sequence according to a gene) may contain four unique phosphorylation sites, and may further have three unique splicing isoforms that do not affect any of the phosphorylation sites. The particular protein can have 48 unique proteoforms (i.e., (3 isoforms)×(2⁴ post-translationally modified forms)=48 unique proteoforms). Accordingly, a plurality of binding targets containing each of the 48 unique proteoforms may be provided to a method of characterizing a binding reagent for one of the four phosphorylation sites or three splice variants.

In other cases, a plurality of binding targets may be provided that contains fewer binding targets than the quantity of binding targets provided in a binding profile. For example, a complex proteome, such as the human proteome, can contain in excess of 2×10⁴ unique proteins, as determined by full-length primary amino acid sequences. A plurality of binding targets may comprise a subset of the proteins of the proteome or another protein-containing system. In some cases, a plurality of binding targets provided to a characterization method for a binding reagent that binds an epitope Θ may comprise all proteins of a proteome that contain the epitope Θ. In particular cases, a plurality of binding targets provided to a characterization method for a binding reagent that binds an epitope Θ may further comprise one or more proteins of a proteome that contain a structural variant of the epitope Θ. For example, a plurality of binding targets may comprise one or more proteins containing epitopes that are single amino acid substitutions of an epitope DTR (i.e., proteins containing epitopes such as DTK, DTA, DKR, DAR, KTR, ATR, etc.). In particular cases, a plurality of binding targets provided to a characterization method for a binding reagent that binds an epitope Θ may further comprise one or more proteins of a proteome that do not contain an epitope Θ and/or a structural variant thereof.

A plurality of binding targets may comprise at least about 10, 20, 50, 100, 500, 10³, 10⁴, 10⁵, 10⁶, 10⁷, 10⁸, 10⁹, or more than 10⁹ unique binding targets, as determined by primary amino acid sequence. Alternatively or additionally, a plurality of binding targets may comprise no more than about 10⁹, 10⁸, 10⁷, 10⁶, 10⁵, 10⁴, 10³, 500, 100, 50, 20, 10, or less than 10 unique binding targets, as determined by primary amino acid sequence. A plurality of binding targets may comprise at least about 10, 20, 50, 100, 500, 10³, 10⁴, 10⁵, 10⁶, 10⁷, 10⁸, 10⁹, or more than 10⁹ unique binding targets, as determined by proteoform. Alternatively or additionally, a plurality of binding targets may comprise no more than about 10⁹, 10⁸, 10⁷, 10⁶, 10⁵, 10⁴, 10³, 500, 100, 50, 20, 10, or less than 10 unique binding targets, as determined by proteoform. A plurality of binding targets may comprise at least about 10, 20, 50, 100, 500, 10³, 10⁴, 10⁵, 10⁶, 10⁷, 10⁸, 10⁹, or more than 10⁹ unique proteins. Alternatively or additionally, a plurality of binding targets may comprise no more than about 10⁹, 10⁸, 10⁷, 10⁶, 10⁵, 10⁴, 10³, 500, 100, 50, 20, 10, or less than 10 unique proteins.

In an aspect, provided herein is a method of characterizing a binding reagent, comprising: a) providing a plurality of binding targets, wherein each binding target of the plurality of binding targets is co-localized with a binding target barcode that is specific to the one and only one species of binding target with which it is co-localized, b) coupling binding reagents of a plurality of binding reagents to the plurality of binding targets, wherein each binding reagent is co-localized with a binding reagent barcode, c) for each binding reagent bound to a binding target of the plurality of binding targets, forming an interaction moiety comprising the binding reagent barcode and the binding target barcode, d) detecting the binding reagent barcode and the binding target barcode for each interaction moiety, and e) for each binding reagent barcode, determining a set of binding target barcodes associated with the binding reagent barcode, thereby determining a set of binding targets bound by the binding reagent attached to the binding reagent barcode.

In another aspect, provided herein is a method of characterizing a binding reagent having a binding specificity for an epitope Θ, comprising: (a) providing a plurality of binding targets, wherein each binding target of the plurality of binding targets is derived from a proteome, wherein a first fraction of binding targets of the plurality of binding targets comprises the epitope Θ, wherein a second fraction of binding targets of the plurality of binding targets does not comprise the epitope Θ, and wherein each unique binding target of the plurality of binding targets is co-localized with a unique binding target barcode moiety, (b) contacting to the plurality of binding targets a plurality of binding reagents, wherein each binding reagent of the plurality of binding reagents is substantially identical, wherein each binding reagent of the plurality of binding reagents has a binding specificity for the epitope Θ, and wherein each individual binding reagent of the plurality of binding reagents is attached to a binding reagent barcode moiety, (c) forming a plurality of interaction moieties, wherein each interaction moiety of the plurality of interaction moieties comprises information from a binding target barcode moiety of the plurality of binding targets and information from the binding reagent barcode moiety, (d) detecting each interaction moiety of the plurality of interaction moieties, and (e) determining for each unique binding target barcode moiety a quantity of interaction moieties of the plurality of interaction moieties containing the information from the unique binding target barcode moiety.

The skilled person will recognize that certain methods provided herein for the screening of binding reagents as well as certain methods provided herein for the characterization of a binding reagent can utilize multiplexed pluralities of binding targets (i.e., pluralities of binding targets comprising two or more unique binding targets). For example, both screening and characterization methods may utilize pluralities of binding targets that contain an epitope Θ in a plurality of sequence contexts. Likewise, certain methods provided herein for the screening of binding reagents as well as certain methods provided herein for the characterization of a binding reagent can utilize a plurality of binding targets in which each binding target has an identical structure. For example, a binding reagent may be selected against a specific protein target utilizing a uniform plurality of binding targets, and the uniform plurality of binding targets may be further utilized to measure the binding affinity of the selected binding reagent for the specific protein target. Accordingly, the skilled person will further recognize that the provided methods of forming, utilizing, and/or detecting barcode moieties provided throughout the present disclosure may be useful for both the screening of binding reagents and the characterization of a binding reagent.

A method of characterizing a binding reagent may be performed utilizing an array of binding targets. An array of binding targets may comprise a plurality of sites, in which one or more binding targets are immobilized at each site of the plurality of sites. In some configurations, each site of a plurality of sites may comprise one and only one binding target (e.g., a single-molecule array). Alternatively, each site of a plurality of sites may comprise two or more binding targets (e.g., a protein microarray). A site comprising two or more binding targets may comprise two or more binding targets having identical molecular structures. A site comprising two or more binding targets may comprise two or more binding targets having identical proteoforms. In some configurations, an array of binding targets can comprise an array of polypeptides. In such configurations, each site of the plurality of sites can comprise one and only one species of polypeptide (e.g., as determined by primary amino acid sequence, as determined by proteoform).

A plurality of binding targets may comprise a plurality of unique primary amino acid sequences, in which each unique primary amino acid sequence is derived from a proteome of an organism. A plurality of proteins may comprise at least about 0.1%, 0.5%, 1%, 5%, 10%, 20%, 30%, 40%, 50%, 60%, 70%, 80%, 90%, 95%, 99%, 99.9%, or more than 99.9% of unique primary amino acid sequences of the proteome of the organism, for example as determined by the genome of the organism. Alternatively or additionally, a plurality of proteins may comprise no more than about 99.9%, 99%, 95%, 90%, 80%, 70%, 60%, 50%, 40%, 30%, 20%, 10%, 5%, 1%, 0.5%, 0.1%, or less than 0.1% of unique primary amino acid sequences of the proteome of the organism.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A method of characterizing a binding reagent may comprise a step of, based upon determined quantities of interaction moieties for each binding target of a plurality of binding targets, determining a binding profile for the binding reagent. After forming a binding profile for a binding reagent, the binding profile may be provided during an analyte characterization assay that utilizes the binding reagent. Methods of forming binding profiles are known in the art. Methods of forming a binding profile provided in U.S. Pat. No. 11,970,693, which is herein incorporated by reference, may be particularly useful. In some cases, a binding profile for a binding reagent may comprise for a set of binding targets (e.g., a set comprising each protein of a proteome as determined by full-length primary amino acid sequence) presence or absence of binding of the binding reagent to each individual binding target of the set of binding targets. In some cases, a binding profile may comprise for a set of binding targets (e.g., a set comprising each protein of a proteome as determined by full-length primary amino acid sequence) a probability of binding of the binding reagent to each individual binding target of the set of binding targets.

A binding profile may be formed by a processor that is configured to form a binding profile based upon detection data provided to the processor by a detection device, as set forth herein. In some cases, determining a binding profile for the binding reagent comprises providing determined quantities of interaction moieties for each binding target of a plurality of binding targets to a computational algorithm. A computational algorithm may comprise a training algorithm or a machine learning algorithm. A computational algorithm may be configured to receive binding data of a binding reagent to plurality of binding targets of a subset of a proteome or a set of structurally diverse proteins and predict a binding behavior (e.g., a presence or absence of binding; a probability of binding, etc.) for the binding reagent to one or more proteins not included in the plurality of binding targets.

### Binding Reagent Selection and Characterization Systems

The present disclosure further provides systems that are configured to perform methods of screening and/or characterizing binding reagents, as set forth herein. A binding reagent may be selected for a particular assay, such as an assay set forth in the section titled “Single-Analyte Assays.” In some configurations, a system may be configured to carry out steps of a method for two or more of: i) selecting a binding reagent from a library of binding reagents, ii) characterizing a binding specificity and/or binding affinity of a binding reagent, and iii) characterizing one or more analytes utilizing a binding reagent (e.g., a binding reagent selected and/or characterized by the system performing the analyte characterization).

It may be advantageous to screen and/or characterize a binding reagent on a same system that performs an analyte characterization assay. The binding characteristics of a binding reagent may be influenced not only by the composition of the binding reagent itself, but also the composition and chemical environment of the system containing the binding reagent. For example, array-based systems (e.g., single-molecule arrays, microarrays, etc.) may utilize solid supports with surface chemistries that are optimized for an entire assay. The chosen surface chemistry of an array may not have the optimal configuration for each reagent of an assay, such as a binding reagent, but may have the best configuration for all of the reagents of the assay collectively. Characterizing a binding reagent in a system that differs from the system in which it will be utilized for an assay may produce a characterization of the binding reagent that does not reflect its behavior in the system for the assay.

Accordingly, a method for screening and/or characterizing a binding reagent, and a method of performing an assay utilizing the binding reagent may be performed on a single system that is configured to perform said methods. A method for screening and/or characterizing a binding reagent, and a method of performing an assay utilizing the binding reagent may utilize common system components to perform said methods. A method for screening and/or characterizing a binding reagent, and a method of performing an assay utilizing the binding reagent may utilize system components that have common configurations. For example, if an assay utilizing a binding reagent is performed with an array provided on a solid support (e.g., a single-molecule array, a microarray, etc.), the binding reagent may be screened and/or characterized on an array with a substantially identical configuration (e.g., shape, geometry, size, site density, binding target density, surface chemistry, architecture, etc.). In another example, if an assay utilizing a binding reagent is performed in a fluid phase, the binding reagent may be screened and/or characterized in a vessel that is substantially identical to the vessel in which the assay is performed (e.g., a 96-well plate, a 384-well plate, etc.).

A system configured to perform a method for screening and/or characterizing a binding reagent and a method of performing an assay utilizing the binding reagent may comprise one or more reagents that are utilized for each of the methods performed on the system. Common reagents can include buffers, rinsing media, binding reagent association media, binding reagent dissociation media, and system maintenance media (e.g., cleaning reagents, storage reagents, etc.). A system may be configured to provide a binding reagent during a method for screening and/or characterizing a binding reagent and a method of performing an assay utilizing the binding reagent in substantially identical chemical environments. Accordingly, a system may comprise one or more environmental control systems, such as pH control, temperature control, and mass flow controllers to provide a medium containing a binding reagent to a plurality of binding targets under substantially uniform conditions.

A method for screening and/or characterizing a binding reagent and a method of performing an assay utilizing the binding reagent may comprise analogous processes. For example, an assay may include incubating a binding reagent with a plurality of binding targets for a time period. In this example, a system may be configured to incubate a binding reagent with a plurality of binding targets for an identical time period during a method of screening and/or characterizing a binding reagent and an assay utilizing the binding reagent. In another example, an assay may include a step of removing unbound binding reagents from contact with a plurality of binding targets with a rinsing medium. In this example, a system may be configured to provide an identical quantity or flow rate of the rinsing medium for a time period for the method of screening and/or characterizing a binding reagent and the assay.

In another aspect, provided herein is a system for screening a library of binding reagents, characterizing a binding reagent, and/or performing an assay utilizing a binding reagent, comprising: a) a vessel comprising a plurality of binding reagents, b) a plurality of binding targets, c) a polymer sequencing device, and d) a fluidic system that is configured to transfer the plurality of binding reagents from the vessel to the plurality of binding targets, and is further configured to transfer binding interaction moieties from the plurality of binding targets to the polymer sequencing device.

A system for performing a method set forth herein may contain one or more fluidic media that facilitate a step of the method. Fluidic media can include a medium for rinsing or removing unbound entities (e.g., unbound binding reagents, dissociated interaction moieties) from contact with a plurality of binding targets, a medium for associating binding reagents to binding targets, a medium for dissociating binding reagents from binding targets, and a medium for transferring moieties to a sequencing device. Useful compositions for fluidic media for binding reagent assays are described in U.S. Patent Publication No. 20240192202A1, which is incorporated herein by reference in its entirety. A fluidic medium may be stored in a reservoir that is in fluidic communication with a fluidics system that is configured to transfer the fluidic medium to one or more other components of a system (e.g., a flow cell, a vessel, an array, a sequencing device, etc.).

A system for performing a method set forth herein may comprise a flow cell. A flow cell may comprise one or more fluidic channels and/or chambers. An array of sites may be disposed in a channel or chamber of a flow cell. In some configurations, an array may be provided to a system, in which a plurality of binding targets is immobilized on the array. In some configurations, a fluidics system may be configured to deliver a plurality of binding targets to a flow cell containing an array of sites, thereby facilitating immobilization of the binding targets on sites of the array of sites.

A system for performing a method set forth herein may comprise one or more vessels that are configured for a fluid phase method. A fluid phase method may comprise one or more steps in which binding targets and binding reagents are suspended or solvated together in a fluidic medium. It may be advantageous to provide binding targets that are attached to a suspensible solid support, thereby facilitating suspension or solvation of the binding targets in a fluidic medium during certain steps of a method set forth herein, and further facilitating separation of the binding targets from the fluidic medium during other steps. Suspensible solid supports can include beads or other particles to which a binding target can be attached. Particularly useful suspensible solid supports may be readily separable from a fluidic medium, such as magnetic particles, electrically-charged particles, or sedimenting particles. A system may be configured to facilitate separation of a suspensible solid support from a fluidic medium, for example by applying a magnetic field, applying an electric field, or centrifuging the fluidic medium. Accordingly, a system set forth herein may further comprise a device that is configured to provide a separating force (e.g., an electromagnet, an electrical field generator, a centrifuge, etc.). A fluidic system of a system for performing a fluid phase method may comprise an automated fluid handling system, such as an autopipette, that is configured to deliver and/or remove fluids from one or more vessels of the system.

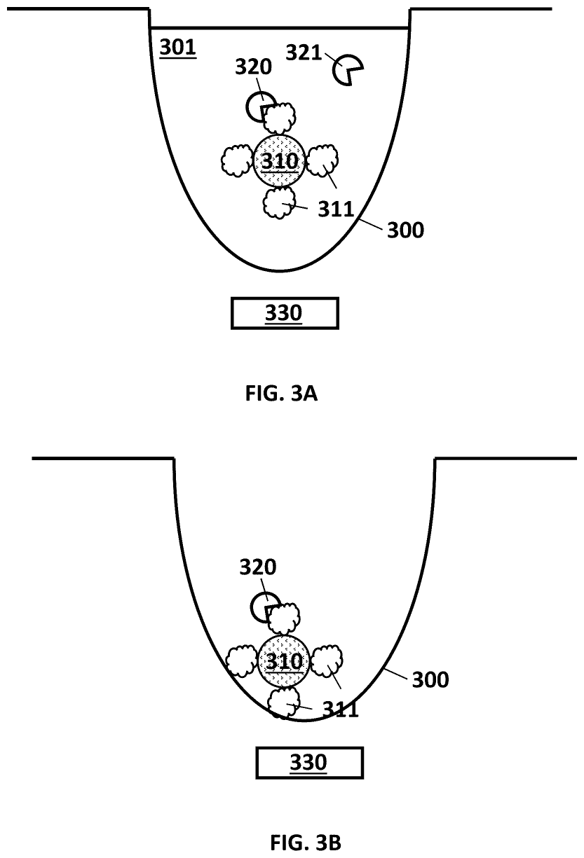

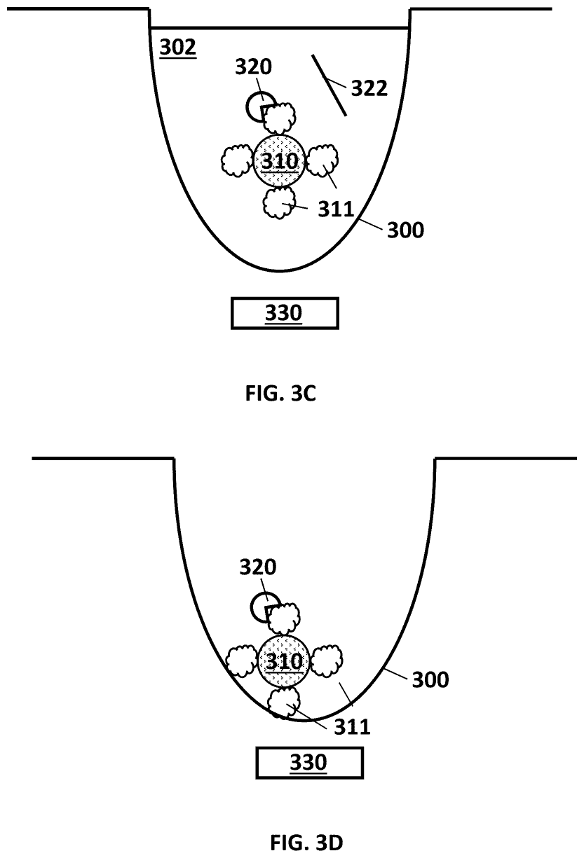

FIGS. 3A-3D illustrate aspects of utilizing a suspensible solid support to perform a method set forth herein. FIG. 3A depicts a vessel 300 comprising a first fluidic medium 301. A suspensible solid support 310 is disposed in the first fluidic medium 301. A plurality of binding targets 311 are attached to the suspensible solid support 310. A plurality of unique binding reagents (320, 321) are also disposed in the first fluidic medium 301. A first binding reagent 320 has bound to a binding target 311 that is attached to the suspensible solid support 310 while a second binding reagent 321 is unbound. The system further comprises a device 330 that is configured to provide a separating force to the suspensible solid support 310. FIG. 3B depicts a second configuration of the system, in which a separating force has been applied to the suspensible solid support 310, thereby transferring the suspensible solid support 310, the binding targets 311, and the bound binding reagent 320 to a surface of the vessel 300. The first fluidic medium 301 has been removed from the vessel 301, thereby removing the unbound binding reagent 321 while retaining the suspensible solid support 310 and any bound or attached entities. The composition of FIG. 3B could be repeatedly rinsed by delivery of a rinsing medium and subsequent removal of the rinsing medium from the vessel 300, with repeated separation of the suspensible solid support 310 from the rinsing medium by utilizing the device 330 before removing the rinsing medium.

FIG. 3C depicts a third configuration, in which a second fluidic medium 302 has been delivered to the vessel 300. A barcode moiety or interaction moiety 322 has been dissociated into the second fluidic medium after association of the first binding reagent 320 to a binding target 311. Analogously to FIG. 3B, FIG. 3D shows how the device 330 may facilitate separation of the suspensible solid support 310 and the plurality of binding targets 311 from the second fluidic medium 302, thereby facilitating removal of the barcode moiety or interaction moiety 322 from the vessel 300 by the removal of the second fluidic medium 302. The second fluidic medium 302 and/or the barcode or interaction moiety 322 may be transferred to a sequencing device (not shown) while the suspensible solid support 310, the plurality of binding targets, and/or the first binding reagent 320 are retained in the vessel 300. Alternatively, if the first binding reagent 320 is attached to a barcode or interaction moiety 322, the first binding reagent 320 may be dissociated from the binding target 311 into the second fluidic medium 302. Subsequently, the barcode or interaction moiety 322 may be separated from the first binding reagent 320, either in the vessel 300 or in a different portion of the fluidics system.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. Optionally, a detection apparatus can be configured to include a waste receptacle to which waste from the vessel is collected. For example, a composition set forth herein can be delivered from the apparatus through an ingress of a flow cell and waste can be removed through an egress of the flow cell to the apparatus.

Methods of screening and characterizing binding reagents set forth herein may involve forming interaction moieties that record binding interactions between binding targets and binding reagents. A system may be configured to serially contact and subsequently separate binding reagents from binding targets, in which each contacting produces a set of interaction moieties. The serial contacting may involve removing a fluid containing binding reagents from contact with the binding targets (e.g., by removing the fluid from a vessel containing the binding targets), then delivering the fluid again to the binding targets, or alternatively delivering a second fluid containing substantially identical binding reagents to the binding targets. Alternatively, the serial contacting may involve a “one-pot” approach, in which binding reagents are associated and dissociated from binding targets without removing the binding reagents from contact with the binding targets (e.g., the binding targets and binding reagents remain in a same fluid during the dissociated phase). A “one-pot” method may produce an interaction moiety containing multiple binding target barcode moieties and/or multiple binding reagent barcode moieties, each of the multiple barcode moieties corresponding to a different cycle of binding reagent binding. Such cycling may produce interaction moieties that facilitate distinction of binding reagents that bind multiple binding targets (e.g., as distinguished by different binding reagent barcode moieties) from binding reagents that repeatedly bind the same binding target (e.g., as distinguished by the same binding reagent barcode moiety).

Accordingly, a system that is configured to perform a “one-pot” method may comprise a device that is configured to produce a dissociated state between binding reagents and binding targets. A system may comprise a thermocycling device (e.g., a Pelletier device, a heat exchanger, etc.) that is configured to cycle a fluidic vessel between a cool state (e.g., about 10° C., 15° C., 20° C., or 25° C.) and a warm state (e.g., about 30° C., 35° C., 40° C., 50° C., 60° C., or 70° C.). Alternatively or additionally, a system may comprise a fluidic device that is configured to modulate a pH or ionic strength of a fluidic medium contacted to binding reagents and binding targets.

A binding reagent for a method set forth herein may be provided in a kit. A kit may comprise a vessel containing the binding reagent. Optionally, the kit provides the binding reagent in a fluidic medium. The fluidic medium containing the binding reagent may be formulated to stabilize the binding reagent during storage. The fluidic medium containing the binding reagent may further comprise one or more excipient reagents (e.g., an anti-aggregant, an anti-flocculant, an antibiotic compound, a cryoprotectant, etc.). A binding reagent for a method of screening and/or characterizing a binding reagent may be provided in a similar or substantially identical fluidic medium to a fluidic medium containing a binding reagent for an assay of analytes. Alternatively, a kit may provide a binding reagent in a non-fluid format, such as a lyophilized or crystallized binding reagent.

A kit provided to a method set forth herein may comprise a library of binding reagents. In some configurations, each unique binding reagent of the library of binding reagents can be provided in a single vessel. In other configurations, the library of binding reagents may be provided in a plurality of vessels. For example, each vessel of a plurality of vessels may comprise a single unique binding reagent, in which each vessel comprise a plurality of binding reagents of the single unique binding reagent. In another example, each vessel of a plurality of vessels may comprise two or more unique binding reagents, but no vessel comprises a complete set of all binding reagents in the library of binding reagents.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

Additionally, provided herein are systems for performing the techniques, reagents, systems, and methods described herein. An example of a system is illustrated in FIG. 5. As shown, the system 500 includes a flowcell 502 that includes an array surface (shown as 504) within the channels of the flow cell upon which individual analyte molecules from a sample may be deposited and immobilized in locations 506 that are individually addressable, and in particular cases are individually optically resolvable from each other using, e.g., fluorescence microscopy or scanning techniques.

The system will also typically include a fluidic delivery system 508 that is configured to deliver different fluids to the flow cell 502 through a series of fluidic lines and utilizing appropriate pumps, valves and other conventional fluid controls. The fluidics system 508 may be fluidically coupled to various sources of fluids and reagents needed to carry out the analysis on the flow cell. For example, as shown, fluidic system 508 is fluidly coupled to a source of a plurality of reagents 510 (shown as a 96 well plate, although any number of different reagent storage systems of varying capacity may be employed) that includes a library of multiple affinity reagents that each have affinity for different characteristics of one or more proteins of interest. Additionally, fluidic system 508 may also be coupled to sources of washing fluids or buffers 512, and removal reagents 514 (for removing bound affinity reagents following detection), as well as any other ancillary fluids and reagents needed for the analysis. Similarly, where flow cells are prepared on the system, the fluidic system may be coupled to sources of different sample materials that are to be analyzed 516 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 518, for detecting and recording fluorescent signals arising from different positions on the array surface. Such detection systems may generally include line scanning confocal fluorescent microscope systems, which are capable of scanning across large array surfaces (as shown by arrow 520) to detect and record fluorescence across such surfaces at reasonably high scan rates.

The overall systems also typically include one or more computers or processors 522 for controlling the operation of the instrument system including the fluidic system 508 (e.g., to sample different sample sources 516, reagent sources 510 and delivery timing and volume of each), and detection system 518, among other functions, and for recording the detected signals received from the detection system 518, e.g. fluorescent signals, and analyzing such signals to identify potential binding by each of the different affinity reagents. Processors 522 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, BioRxiv, DOI: 10.1101/2021.10.11.463967, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 522 can be an electronic device of a detection system, the electronic device being integral to the detection system or remotely located with respect to the detection system. The computer system 522 includes a computer processing unit (CPU, also “processor” and “computer processor” herein), which can be a single core or multi core processor, or a plurality of processors for parallel processing. The computer system 522 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 522 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 522, can implement a peer-to-peer network, which may enable devices coupled to the computer system 522 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 522 can be included in the circuit. In some cases, the circuit is an application specific integrated circuit (ASIC).

The storage unit can store files, such as drivers, libraries and saved programs. The storage unit can store user data, e.g., user preferences and user programs. The computer system 522 in some cases can include one or more additional data storage units that are external to the computer system 522, such as located on a remote server that is in communication with the computer system 522 through an intranet or the Internet.

The computer system 522 can communicate with one or more remote computer systems through the network. For instance, the computer system 522 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 522 via the network.

Methods as described herein can be implemented by way of machine (e.g., computer processor) executable code stored on an electronic storage location of the computer system 522, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 522, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 522 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Single-Analyte Assays

The present disclosure further provides compositions, apparatus and methods for detecting one or more proteins. A protein can be detected using one or more affinity agents having binding affinity for the protein, as identified by a method of binding reagent screening or characterization set forth herein. Binding reagents screened or characterized by a method set forth herein may be especially useful for methods of analyte characterization described in U.S. patent application Ser. No. 19/338,819, which is herein incorporated by reference in its entirety. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in US Pat. No. 10,473,654 or U.S. Pat. No. 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. Nos. 10,473,654 or U.S. Pat. No. 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or U.S. Pat. No. 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g. the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g. technology developed by SomaLogic or Palamaedrix) may also be useful for detecting proteins.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. No. 11,203,612 or U.S. Pat. No. 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. Pat. No. 11,203,612, which is incorporated herein by reference. Non-covalent attachment can be mediated by receptor-ligand interactions (e.g. (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. Nos. 11,203,612 and 11,505,796, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different analytes can be attached to different unique identifiers (e.g. proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g. ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g. an address in an array) using any of a variety of means. The attachment can be covalent or non-covalent. Exemplary covalent attachments include chemical linkers such as those achieved using click chemistry or other linkages known in the art or described in U.S. Pat. No. 11,203,612 or U.S. Pat. No. 11,505,796 or US Pat. App. Pub. No 2023/0167488 A1, each of which is incorporated herein by reference. Non-covalent attachment can be mediated by receptor-ligand interactions (e.g. (strept) avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in U.S. Pat. No. 11,203,612 or U.S. Pat. No. 11,505,796 or US Pat. App. Pub. No 2023/0167488 A1, each of which is incorporated herein by reference.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexamethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

An array of analytes may be provided with a characterized or characterizable analyte-binding site occupancy. The analyte-binding site occupancy can be measured as the fraction or percentage of analyte-binding sites of a plurality of analyte-binding sites containing an attached analyte. An array of analytes may be provided with an analyte-binding site occupancy of at least about 10%, 20%, 25%, 30%, 35%, 40%, 45%, 50%, 60%, 70%, 80%, 90%, 95%, 99%, 99.9%, or more than 99.9%. Alternatively or additionally, an array of analytes may be provided with an analyte-binding site occupancy of no more than about 99.9%, 99%, 95%, 90%, 80%, 70%, 60%, 50%, 45%, 40%, 35%, 30%, 25%, 20%, 10%, or less than 10%.

An array of analytes may be provided with a fraction or percentage of individual sites that each contain one and only one analyte. The fraction or percentage may be calculated relative to all other sites in the array including, but not limited to, those containing no analyte and those containing multiple analytes. Preferably, an array of analytes may be provided with super Poisson loading of single analytes (i.e., a fraction or percentage of attachments sites containing one and only one analyte exceeding 37%). An array of analytes may be provided with at least about 10%, 20%, 25%, 30%, 35%, 37%, 40%, 45%, 50%, 60%, 70%, 80%, 90%, 95%, 99%, 99.9%, or more than 99.9% of analyte-binding sites containing one and only one analyte. Alternatively or additionally, an array of analytes may be provided with no more than about 99.9%, 99%, 95%, 90%, 80%, 70%, 60%, 50%, 45%, 40%, 35%, 30%, 25%, 20%, 10%, or less than 10% of analyte-binding sites containing one and only one analyte.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10², 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or U.S. Pat. No. 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. No. 11,203,612 or U.S. Pat. No. 11,505,796; or US Pat. App. Pub. No. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g. SNAP) or population thereof has a minimum, maximum or average length of at least about 50 nm, 100 nm, 250 nm, 500 nm, 1 micron, 5 micron or more. Alternatively or additionally, a retaining component (e.g. SNAP) or population thereof has a minimum, maximum or average length of no more than about 5 micron, 1 micron, 500 nm, 250 nm, 100 nm, 50 nm, or less.

Optionally, a retaining component (e.g. SNAP) or population thereof has a minimum, maximum or average volume of at least about 1 micron³, 10 micron³, 100 micron³, 1 mm³ or more. Alternatively or additionally, a retaining component (e.g. SNAP) or population thereof has a minimum, maximum or average volume of no more than about 1 mm³, 100 micron³, 10 micron³, 1 micron³ or less.

Optionally, the minimum, maximum or average area (i.e. footprint) for a retaining component (e.g. SNAP) is at least about 10 nm², 100 nm², 1 micron², 10 micron², 100 micron², 1 mm² or more. Alternatively or additionally, the minimum, maximum or average area for a retaining component (e.g. SNAP) footprint is at most about 1 mm², 100 micron², 10 micron², 1 micron², 100 nm², 10 nm², or less. The footprint of a retaining component (e.g. SNAP) may have a regular shape or an approximately regular shape, such as triangular, square, rectangular, circular, ovoid, or polygonal shape.

A structured nucleic acid particle (e.g. having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g. protein), paratope, affinity moiety (e.g. antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or U.S. Pat. No. 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

An retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), peptides (e.g. proteins, polypeptides or oligopeptides), amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17): 3982 (2020); Zheng, et al. or Chem. Soc. Rev. 44, 4091-4130 (2015), each of which is incorporated herein by reference.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. No. 11,203,612 or U.S. Pat. No. 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspensibility of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH⁴⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN, CO₃²⁻, CrO₄²⁻, Cr₂O₇²⁻, HPO₄²⁻, SO₄²⁻, SO₃²⁻, PO₄⁻³, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio) butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl)phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo [2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese (III)-tetrakis(4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

A fluidic medium may be provided at, heated to, cooled to, or maintained at a temperature of at least about-80 degrees Celsius (° C.), −50° C., −10° C., −5° C., 0° C., 5° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 60° C., 70° C., 80° C., 90° C., 95° C., or more than 95° C. Alternatively or additionally, a fluidic medium may be provided at, heated to, cooled to, or maintained at a temperature of no more than about 95° C., 90° C., 80° C., 70° C., 60° C., 50° C., 45° C., 40° C., 35° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 5° C., 0° C., −5° C., −10° C., −50° C., −80° C., or less than −80° C.

A fluidic medium may be provided at or adjusted to a pH of at least about 0.0, 0.5, 1.0, 1.5, 2.0, 2.5, 3.0, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, 9.0, 9.5, 10.0, 10.5, 11.0, 11.5, 12.0, 12.5, 13.0, 13.5, 14.0, or more than 14.0. Alternatively or additionally, a fluidic medium may be provided at or adjusted to a pH of no more than about 14.0, 13.5, 13.0, 12.5, 12.0, 11.5, 11.0, 10.5, 10.0, 9.5, 9.0, 8.5, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.5, 5.0, 4.5, 4.0, 3.5, 3.0, 2.5, 2.0, 1.5, 1.0, 0.5, or less than 0.5

A component of a fluidic medium may be provided at or adjusted to a molar concentration of at least about 0.0001 moles per liter (M), 0.001M, 0.01M, 0.02M, 0.03M, 0.04M, 0.05M, 0.06M, 0.07M, 0.08M, 0.09M, 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 7M, 8M, 9M or more than 10M. Alternatively or additionally, a component of a fluidic medium may be provided at or adjusted to a molar concentration of no more than about 10 M, 9M, 8M, 7M, 6M, 5.9M, 5.8M, 5.7M, 5.6M, 5.5M, 5.4M, 5.3M, 5.2M, 5.1M, 5.0M, 4.9M, 4.8M, 4.7M, 4.6M, 4.5M, 4.4M, 4.3M, 4.2M, 4.1M, 4.0M, 3.9M, 3.8M, 3.7M, 3.6M, 3.5M, 3.4M, 3.3M, 3.2M, 3.1M, 3.0M, 2.9M, 2.8M, 2.7M, 2.6M, 2.5M, 2.4M, 2.3M, 2.2M, 2.1M, 2.0M, 1.9M, 1.8M, 1.7M, 1.6M, 1.5M, 1.4M, 1.3M, 1.2M, 1.1M, 1.0M, 0.9M, 0.8M, 0.7M, 0.6M, 0.5M, 0.4M, 0.3M, 0.2M, 0.1M, 0.09M, 0.08M, 0.07M, 0.06M, 0.05M, 0.04M, 0.03M, 0.02M, 0.01M, 0.001M, 0.001M, or less than about 0.001M.

A component of a fluidic medium may be provided at or adjusted to a weight or volumetric percentage of at least about 0.0001%, 0.001%, 0.005%, 0.01%, 0.05%, 0.1%, 0.5%, 1%, 2%, 3%, 4%, 45%, 6%, 7%, 8%, 9%, 10%, 20%, 30%, 40%, 50%, or more than 50%.

Alternatively or additionally, a component of a fluidic medium may be provided at or adjusted to a weight or volumetric percentage of no more than about 50%, 40%, 30%, 20%, 10%, 9%, 8%, 7%, 6%, 5%, 4%, 3%, 2%, 1%, 0.5%, 0.1%, 0.05%, 0.01%, 0.005%, 0.001%, 0.0001%, or less than 0.0001%.

A plurality of proteins can be characterized in terms of total protein mass. The total mass of protein in a liter of plasma has been estimated to be 70 g and the total mass of protein in a human cell has been estimated to be between 100 μg and 500 μg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 μg, 10 μg, 100 μg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 μg, 10 μg, 1 μg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076× 1023 molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

Proteoforms can differ with regard to presence or absence of a post-translational modification, type of post-translational modification present, location of a post-translational modification, number of post-translational modifications present or combination thereof.

A method of the present disclosure can include a step of removing post-translational moieties from post-translationally modified amino acids, thereby forming standard amino acids. In some cases, an enzyme can be used to remove a post-translational moiety from an amino acid.

An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020); DOI: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

1. A method of selecting a binding reagent having a binding specificity for an epitope Θ, comprising:
(a) providing a plurality of binding targets, wherein each binding target of the plurality of binding targets comprises the epitope Θ, and wherein each binding target of the plurality of binding targets is co-localized with a unique binding target barcode moiety;
(b) providing a library of binding reagents wherein each binding reagent of the library of binding reagents differs from each other binding reagent of the library of binding reagents with respect to binding specificity, and wherein each binding reagent of the library of binding reagents is attached to a unique binding reagent barcode moiety;
(c) combining the plurality of binding targets with the library of binding reagents, thereby coupling binding reagents of the library of binding reagents to binding targets of the plurality of binding targets;
(d) forming a plurality of interaction moieties, wherein each interaction moiety of the plurality of interaction moieties comprises information from a binding target barcode moiety of a binding target of the plurality of binding targets and information from the binding reagent barcode moiety; and
(e) detecting two or more interaction moieties containing information from a same binding reagent barcode moiety, thereby selecting the binding reagent of the library of binding reagents having a binding specificity for the epitope Θ.

2. The method of claim 1, wherein step (e) comprises detecting each interaction moiety of the plurality of interaction moieties.

3. The method of claim 2, further comprising: (i) identifying a first set of interaction moieties, wherein each interaction moiety of the first set of interaction moieties comprises information from a first same binding reagent identification moiety of a first binding reagent of the library of binding reagents; (ii) identifying a second set of interaction moieties, wherein each interaction moiety of the second set of interaction moieties comprises information from a second same binding reagent identification moiety of a second binding reagent of the library of binding reagents; and (iii) determining which of the first set of interaction moieties and the second set of interaction moieties contains a greater quantity of interaction moieties, thereby selecting the binding reagent of the library of binding reagents having a binding specificity for the epitope Θ.

4. The method of claim 1, wherein forming an interaction moiety of the plurality of interaction moieties comprises attaching a binding target barcode moiety co-localized with a binding target to a binding reagent barcode moiety that is attached to a binding reagent of the library of binding reagents.

5. The method of claim 4, wherein attaching the binding target barcode moiety co-localized with the binding target to the binding reagent barcode moiety that is attached to the binding reagent of the library of binding reagents comprises ligating the binding target barcode moiety co-localized with the binding target to the binding reagent barcode moiety that is attached to the binding reagent of the library of binding reagents.

6. The method of claim 5, wherein the binding reagent barcode moiety and the binding target barcode moiety comprise peptides.

7. The method of claim 5, wherein the binding reagent barcode moiety and the binding target barcode moiety comprise nucleic acids.

8. The method of claim 4, wherein attaching the binding target barcode moiety co-localized with the binding target to the binding reagent barcode moiety that is attached to the binding reagent of the library of binding reagents comprises hybridizing a nucleic acid strand of the binding target barcode moiety co-localized with the binding target to a nucleic acid strand of the binding reagent barcode moiety that is attached to the binding reagent of the library of binding reagents.

9. The method of claim 8, further comprising extending the nucleic acid strand of the binding target barcode moiety or the nucleic acid strand of the binding reagent barcode moiety by polymerase extension.

10. The method of claim 1, further comprising dissociating the plurality of interaction moieties from the plurality of binding targets and the library of binding reagents.

11. The method of claim 10, wherein dissociating the plurality of interaction moieties from the plurality of binding targets and the library of binding reagents comprises cleaving a labile linking group attached to an interaction moiety of the plurality of interaction moieties.

12. The method of claim 1, wherein the plurality of analytes is attached to a solid support.

13. The method of claim 12, wherein the solid support is suspensible in a fluidic medium.

14. The method of claim 12, wherein the solid support is not suspensible in a fluidic medium.

15. The method of claim 1, wherein the plurality of analytes is not attached to a solid support.

16. A method of characterizing a binding reagent having a binding specificity for an epitope Θ, comprising:
(a) providing a plurality of binding targets, wherein each binding target of the plurality of binding targets is derived from a proteome, wherein a first fraction of binding targets of the plurality of binding targets comprises the epitope Θ, wherein a second fraction of binding targets of the plurality of binding targets does not comprise the epitope Θ, and wherein each unique binding target of the plurality of binding targets is co-localized with a unique binding target barcode moiety;
(b) contacting to the plurality of binding targets a plurality of binding reagents, wherein each binding reagent of the plurality of binding reagents is substantially identical, wherein each binding reagent of the plurality of binding reagents has a binding specificity for the epitope Θ, and wherein each individual binding reagent of the plurality of binding reagents is attached to a binding reagent barcode moiety;
(c) forming a plurality of interaction moieties, wherein each interaction moiety of the plurality of interaction moieties comprises information from a binding target barcode moiety of the plurality of binding targets and information from the binding reagent barcode moiety;
(d) detecting each interaction moiety of the plurality of interaction moieties; and
(e) determining for each unique binding target barcode moiety a quantity of interaction moieties of the plurality of interaction moieties containing the information from the unique binding target barcode moiety.

17.-30. (canceled)

31. A system for characterizing a binding reagent, comprising:
a) a vessel, wherein the vessel comprises a fluidic medium comprising:
(i) a plurality of binding reagents; and
(ii) a plurality of binding targets, wherein each binding target of the plurality of binding targets is co-localized with a unique binding target barcode moiety;
b) a polymer sequencing device; and
c) a fluidic system that is configured to transfer interaction moieties from the vessel to the polymer sequencing device.
